(19) United States
(12) Patent Application Publication
(10) Pub. No.: US 2025/0340842 A1
(43) Pub. Date: Nov. 6, 2025

(54) METHOD FOR EXPANDING GAMMA DELTA T CELLS

(71) Applicant: GAMMADELTA THERAPEUTICS LTD, London (GB)

(72) Inventors: Andre Goncalo do Espirito Santo SIMOES, London (GB); Istvan KOVACS, London (GB); Oliver NUSSBAUMER, London (GB); Sara TAMAGNO, London (GB)

(73) Assignee: GAMMADELTA THERAPEUTICS LTD, London (GB)

(21) Appl. No.: 18/854,458

(22) PCT Filed: Apr. 4, 2023
(86) PCT No.: PCT/IB2023/053432

§ 371 Date: Oct. 4, 2024

(30) Foreign Application Priority Data
Apr. 4, 2022 (GB) 2204926.6

(51) Int. Cl. C12N 5/0783; A61K 40/11; A61K 40/31; A61K 40/42

(52) U.S. Cl. CPC C12N 5/0638; A61K 40/11; A61K 40/31; A61K 40/4255; C12N 2501/2315; C12N 2501/515; C12N 2510/00

(57) ABSTRACT

The present disclosure provides methods for expanding γδ T cells (e.g., vδ1 T cells), wherein the cells are contacted with IL-15. In some aspects, the cells are not contacted with IL-4. In some aspects, the cells are engineered, e.g., to express a chimeric antigen receptor. Further provided are populations of expanded and/or engineered γδ T cells and methods of using the same.

### CROSS REFERENCE TO RELATED APPLICATIONS

This PCT application claims the priority benefit of Great Britain Application No. 2204926.6, filed Apr. 4, 2022; which is incorporated herein by reference in its entirety.

### FIELD OF THE INVENTION

The invention relates to methods for expanding γδ T cells and optionally engineering said cells. Expanded and engineered γδ T cells produced according to the methods described herein find use in the treatment of cancer, in particular solid tumours. The present invention also relates to both individual cells and populations of cells produced by the methods described herein.

### BACKGROUND OF THE INVENTION

Gamma delta T cells (γδ T cells) represent a subset of T cells that express on their surface a distinct, defining γδ T-cell receptor (TCR). This TCR is made up of one gamma (γ) and one delta (δ) chain. Human γδ TCR chains are selected from three main δ chains, Vδ1, Vδ2 and Vδ3 and six γ chains. Human γδ T cells can be broadly classified based on their TCR chains, as certain γ and δ types are found on cells more prevalently, though not exclusively, in one or more tissue types. For example, most blood-resident γδ T cells express a Vδ2 TCR, for example Vγ9Vδ2, whereas this is less common among tissue-resident γδ T cells, which more frequently use Vδ1 in skin and Vγ4 in the gut.

Vδ1 γδ T cells are a subset of innate T cells defined by expression of T cell receptors composed of a γ chain paired to a Vδ1 chain. In mice, Vδ1 γδ T cells are predominantly tissue resident where they are highly protective against a broad spectrum of carcinomas by mediating anti-tumour responses via pattern and natural cytotoxicity receptor recognition. Similarly, in humans, Vδ1 γδ T cells predominantly reside within epithelial tissues, mediate target cell recognition that is not MHC restricted and are not allo-HLA reactive. HLA matching of patients is therefore not required for γδ T cell adoptive cell therapies. The innate Vδ1 γδ T cell biology which enables antigen independent tumour recognition, lack of necessity for HLA matching, and inherent migration to and residence in human tissues makes Vδ1 γδ T cells an attractive platform for cellular therapy.

There is therefore a need for methods to efficiently expand γδ T cells to allow their adaptation as therapies, e.g. as adoptive T cell therapies, and for methods which have the potential to provide allogeneic ‘off-the-shelf’ chimeric antigen receptor-expressing γδ T cell therapies, such as for the treatment of solid tumours.

WO2015189356 describes a composition for expanding lymphocytes obtained from a sample obtained by aphaeresis comprising at least two types of cytokines selected from IL-2, IL-15 and IL-21. WO2016198480 describes methods of expanding Vδ2-TCRγδ+ T cells, particularly those derived from blood samples, using a two-step culture method comprising culturing cells in a first medium comprising a T cell mitogen and Interleukin-4 (IL-4) and then a second medium comprising a T cell mitogen and Interleukin-15 (IL-15). WO2016081518 describes methods for expanding engineered and non-engineered γδ T cell populations using an antibody that binds to the delta chain of the TCR.

While these disclosures go some way towards addressing the above-mentioned problem, there remains a need for improved methods of expanding γδ T cells, in particular to produce γδ T cells that are effective at targeting solid tumours.

### SUMMARY OF THE INVENTION

According to a first aspect of the invention, there is provided a method for expanding γδ T cells, wherein said method comprises the steps of:
(1) preparing a composition enriched for γδ T cells by depleting αβ T cells from a sample obtained from a subject which comprises γδ T cells;
(2) culturing the composition enriched for γδ T cells in the presence of:
   (i) an anti-CD3 antibody or fragment thereof; and
   (ii) Interleukin-15 (IL-15), in the absence of Interleukin-4 (IL-4) (e.g., wherein IL-4 is not added at all during the method),
   from the first day of said culturing; and
(3) isolating the cell population cultured from the composition

According to a further aspect of the invention, there is provided a method for engineering γδ T cells, said method comprising the steps of:
(i) preparing a composition enriched for γδ T cells using the method as defined herein;
(ii) transducing the composition with an exogenous nucleic acid for expression in the γδ T cells; and
(iii) culturing the transduced composition to expand the engineered γδ T cells.

According to a further aspect of the invention, there is provided an expanded γδ T cell population obtainable, such as obtained, by the method as defined herein.

According to a further aspect of the invention, there is provided a pharmaceutical composition comprising the expanded γδ T cell population as defined herein.

According to a further aspect of the invention, there is provided the expanded γδ T cell population or the pharmaceutical composition as defined herein, for use as a medicament.

According to a further aspect of the invention, there is provided the expanded γδ T cell population or the pharmaceutical composition as defined herein, for use in the treatment of cancer.

In some aspects, the expanded γδ T cells are capable of in vivo cytotoxicity for at least about 7 days, at least about 8 days, at least about 9 days, at least about 10 days, at least about 11 days, at least about 12 days, at least about 13 days, at least about 14 days, at least about 15 days, at least about 16 days, at least about 17 days, at least about 18 days, at least about 19 days, at least about 20 days, or at least about 21 days. In some aspects, the expanded γδ T cells are capable of in vivo cytotoxicity for at least about 14 days.

### BRIEF DESCRIPTION OF THE FIGURES

FIG. 1 pictorially represents the Method 1 and Method 2 expansion protocols described in the Examples.

FIG. 2 graphically represents the cell yield at harvest for cell products generated using either the Method 1 or Method 2 process.

FIG. 3 graphically represents the analysis of immune cell composition by flow cytometry.

FIG. 4 graphically represents the analysis of CD56 expression by flow cytometry in γδ T cell products generated by Method 1 or 2.

FIG. 5 graphically represents the comparison of cell surface marker expression in Vδ1 T cell products generated by Methods 1 or 2.

FIG. 6 graphically represents the comparison of chemokine receptor expression in Vδ1 T cell products generated by Methods 1 or 2.

FIG. 7 graphically represents the deep phenotype analysis of cell products generated by Methods 1 or 2. FIG. 7A is a volcano plot showing differentially expressed cell surface markers between Vδ1 T cell products generated by Methods 1 or 2. FIG. 7B is a pie graph of the top signalling pathways associated with the significantly differentially expressed cell surface markers between Vδ1 T cell products generated by Methods 1 or 2.

FIGS. 8A-8C graphically represent a comparison of secreted soluble factors in γδ T cell products generated by Method 1 or 2. FIG. 8A is a principal component analysis, FIG. 8B is a comparison of key secreted cytokines, and FIG. 8C is a comparison of chemokines.

FIG. 9 graphically represents total cell recovery and viability post-cryopreservation for cell products generated using Methods 1 or 2.

FIG. 10 graphically represents cytotoxicity of cell products generated by Methods 1 or 2 post-cryopreservation against lung cancer cell lines (NCI-H226 and A549), gastric cancer cell line (GSU), colorectal cell line (HT-29), ovarian adenocarcinoma cell line (OvCAR3), skin cancer cell line (A375) and pancreatic cancer cell line (CAPAN2).

FIGS. 11A-11B graphically represent chimeric antigen receptor (CAR) expression in cells products generated by Methods 1 or 2. FIG. 11A is a comparison of CAR expression in cell products generated by Methods 1 or 2. FIG. 11B shows representative flow plots of CAR expression in one donor in cell products generated by Methods 1 or 2.

FIG. 12 graphically represents functionality of the CAR engineered cell products generated by Methods 1 or 2 in a cytotoxicity assay against ovarian adenocarcinoma cell line (OvCAR3) and mesothelin engineered lung cancer cell line (A549-Meso).

FIGS. 13A-13B graphically represent tumour control and proliferation of CAR engineered cell products generated by Methods 1 or 2 upon repeated tumour challenging. FIG. 13A shows tumour control upon repeated challenging every 3/4 days with OVCAR3 (top) or A549-Meso (bottom). FIG. 13B illustrates CAR engineered cell products expansion during repeated tumour challenging assay with OVCAR3 (top) or A549-Meso (bottom).

### DETAILED DESCRIPTION OF THE INVENTION

Previous methods have been described for expanding γδ T cells derived from haematopoietic samples such as peripheral blood. However, while such methods provide γδ T cells with proven efficacy against multiple haematological targets, limited activity has been shown against solid tumour targets. The present invention provides a simplified method for expanding γδ T cells (particularly those derived from blood samples) which produces equivalent or better total cell fold expansion and surprising efficacy against solid malignancies.

Therefore, according to a first aspect of the invention, there is provided a method for expanding γδ T cells, wherein said method comprises the steps of:
(1) preparing a composition enriched for γδ T cells by depleting αβ T cells from a sample obtained from a subject which comprises γδ T cells;
(2) culturing the composition enriched for γδ T cells in the presence of:
   (i) an anti-CD3 antibody or fragment thereof; and
   (ii) Interleukin-15 (IL-15), in the absence of Interleukin-4 (IL-4),
   from the first day of said culturing (e.g., wherein IL-4 is not added at all during the method); and
(3) isolating the cell population cultured from the composition.

In certain embodiments, the sample (i.e. the starting sample) is human. The invention finds particular use with γδ T cells derived from blood or fractions therefrom. It has surprisingly been found that methods of the invention are able to expand γδ T cells derived from haematological samples and yet which have improved cytotoxicity against solid tumours.

Therefore, in one embodiment the sample is a haematopoietic sample or a fraction thereof. In a further embodiment, the sample is selected from peripheral blood, umbilical cord blood, lymphoid tissue, thymus, bone marrow, spleen, node tissue or fractions thereof, in particular peripheral blood or a fraction thereof. In a yet further embodiment, the sample consists of peripheral blood mononuclear cells (PBMCs) or low density mononuclear cells (LDMCs).

In an alternative embodiment, the sample is a non-haematopoietic tissue. References herein to “non-haematopoietic tissues” or “non-haematopoietic tissue sample” include skin (e.g. human skin) and gut (e.g. human gut). Non-haematopoietic tissue is a tissue other than blood, bone marrow, or thymus tissue. In one embodiment, the non-haematopoietic tissue sample is skin (e.g. human skin). In a further embodiment, the non-haematopoietic tissue sample is gut or gastrointestinal tract (e.g. human gut or human gastrointestinal tract). In some embodiments, the non-haematopoietic tissue sample is skin (e.g. human skin), which can be obtained by methods known in the art.

Alternatively, the non-haematopoietic tissue sample is selected from: gastrointestinal tract (e.g. colon or gut), mammary gland, lung, prostate, liver, spleen, pancreas, uterus, vagina and other cutaneous, mucosal or serous membranes.

The sample may be a cancer tissue sample, e.g. from a tumour of the breast or prostate, in particular a human cancer tissue sample. In other embodiments, the sample is not obtained from cancer tissue (e.g. a tissue without a substantial number of tumour cells). For example, the sample may be from a region of skin (e.g. healthy skin) separate from a nearby or adjacent cancer tissue. Thus, in some embodiments, the γδ T cells are not obtained from human cancer tissue.

In one embodiment the sample has been obtained from a human. In an alternative embodiment, the sample has been obtained from a non-human animal subject.

Methods for obtaining such tissues are known in the art. Examples of such methods include scalpel explant or punch biopsy and may vary in size according to the method. In some embodiments, the non-haematopoietic tissue sample is obtained by punch biopsy.

The methods described herein are performed outside the human or animal body, i.e. they are in vitro and/or ex vivo. Thus, in one embodiment the methods described herein are in vitro methods. In a further embodiment, the methods described herein are ex vivo methods.

As used herein, references to “expanded”, “expanded population” or “expanded γδ T cells” includes populations of cells which are larger or contain a larger number of cells than a non-expanded population. Such populations may be large in number, small in number or a mixed population with the expansion of a proportion or particular cell type within the population. It will be appreciated that the term “expansion step” refers to processes which result in expansion or an expanded population. Thus, expansion or an expanded population may be larger in number or contain a larger number of cells compared to a population which has not had an expansion step performed or prior to any expansion step. It will be further appreciated that any numbers indicated herein to indicate expansion (e.g. fold-increase or fold-expansion) are illustrative of an increase in the number or size of a population of cells or the number of cells and are indicative of the amount of expansion.

In one preferred embodiment, the γδ T cells expanded by the methods defined herein comprise a population of Vδ1 T cells.

In other embodiments, the composition enriched for γδ T cells comprises NK cells. As described herein, step (i) comprises depletion of αβ T cells, i.e. the composition enriched for γδ T cells is prepared by depletion of αβ T cells. In a further embodiment, preparing a composition enriched for γδ T cells according to step (i) comprises depletion of αβ T cells from a mixed cell population obtained from a starting sample, such as a haematopoietic sample as described herein. The presence of NK cells in the composition may be advantageous as these cells are also effective cytotoxic cells.

NK cells (also known as large granular lymphocytes (LGL)) are cytotoxic lymphocytes of the innate immune system. They provide rapid responses to e.g. virus-infected cells and tumour cells independently of MHC expression on the surface of the target cell. Therefore, similarly to γδ T cells, the recognition of target cells by NK cells is not MHC restricted and they are not allo-HLA reactive, meaning HLA matching of patients is not required for NK cell-based therapies.

In some embodiments, step (i) (i.e. preparing a composition enriched for γδ T cells) additionally, or alternatively, comprises positively selecting γδ+ T cells from a sample obtained from a subject which comprises γδ T cells.

In one embodiment, the method comprises freezing the expanded γδ T cells. Such frozen expanded γδ T cells may subsequently be thawed for downstream processing (such as further culturing and expansion steps) and/or use (such as therapeutic use). Freezing allows the easy transport and long-term storage of expanded γδ T cells and is well known in the art. Therefore, a method that provides for cells that show good viability and activity after freezing and thawing is advantageous, and not all expansion methods yield such cells.

In one embodiment, the composition of γδ T cells is derived from a single donor. In an alternative embodiment, the composition is derived from multiple donors, i.e., the composition is a ‘pooled’ composition.

In one embodiment the single or multiple donors may comprise a subject which is to be treated with the cell populations or compositions of the invention. Alternatively, the single or multiple donors do not comprise a subject which is to be treated with the cell populations or compositions of the invention.

### Culture Conditions

The present method cultures the γδ T cells in the presence of a TCR agonist, in particular an anti-CD3 antibody or fragment thereof. Said antibody may specifically bind to CD3. Preferred antibody clones include anti-CD3 antibodies such as OKT-3 and UCHT-1 clones. In some aspects, retronectin is used in combination with OKT-3.

The term “antibody” includes any antibody protein construct comprising at least one antibody variable domain comprising at least one antigen binding site (ABS). Antibodies include, but are not limited to, immunoglobulins of types IgA, IgG, IgE, IgD, IgM (as well as subtypes thereof). The overall structure of Immunoglobulin G (IgG) antibodies assembled from two identical heavy (H)-chain and two identical light (L)-chain polypeptides is well established and highly conserved in mammals (Padlan (1994) Mol. Immunol. 31:169-217).

A fragment of the antibody (which may also be referred to as “antibody fragment”, “immunoglobulin fragment”, “antigen-binding fragment” or “antigen-binding polypeptide”) as used herein refers to a portion of an antibody (or constructs that contain said portion) that specifically binds to the target, the CD3 protein that is part of the T cell receptor (TCR) complex (e.g. a molecule in which one or more immunoglobulin chains is not full length, but which specifically binds to the target). Examples of binding fragments encompassed within the term antibody fragment include:
(i) a Fab fragment (a monovalent fragment consisting of the VL, VH, CL and CH1 domains);
(ii) a F(ab′)2 fragment (a bivalent fragment consisting of two Fab fragments linked by a disulphide bridge at the hinge region);
(iii) a Fd fragment (consisting of the VH and CH1 domains);
(iv) a Fv fragment (consisting of the VL and VH domains of a single arm of an antibody);
(v) a single chain variable fragment, scFv (consisting of VL and VH domains joined, using recombinant methods, by a synthetic linker that enables them to be made as a single protein chain in which the VL and VH regions pair to form monovalent molecules);
(vi) a VH (an immunoglobulin chain variable domain consisting of a VH domain);
(vii) a VL (an immunoglobulin chain variable domain consisting of a VL domain);
(viii) a domain antibody (dAb, consisting of either the VH or VL domain);
(ix) a minibody (consisting of a pair of scFv fragments which are linked via CH3 domains); and
(x) a diabody (consisting of a noncovalent dimer of scFv fragments that consist of a VH domain from one antibody connected by a small peptide linker to a VL domain from another antibody).

“Specificity” refers to the number of different types of antigens or antigenic determinants to which a particular antibody or fragment thereof can bind. The specificity of an antibody is the ability of the antibody to recognise a particular antigen as a unique molecular entity and distinguish it from another. An antibody that “specifically binds” to an antigen or an epitope is a term well understood in the art. A molecule is said to exhibit “specific binding” if it reacts more frequently, more rapidly, with greater duration and/or with greater affinity with a particular target antigen or epitope, than it does with alternative targets. An antibody “specifically binds” to a target antigen or epitope if it binds with greater affinity, avidity, more readily, and/or with greater duration than it binds to other substances. An antibody (or fragment thereof) may be considered to specifically bind to a target if the binding is statistically significant compared to a non-relevant binder.

In one embodiment, the anti-CD3 antibody or fragment thereof is OKT3. This antibody, also known as Muromonab-CD3, is a murine monoclonal antibody targeting an epitope located on the CD3epsilon chain.

In one embodiment, the anti-CD3 antibody or fragment thereof is in a soluble or immobilized form. For example, the antibody or fragment thereof may be administered to the composition in a soluble form. Alternatively, the antibody or fragment thereof may be administered to the composition when the antibody or fragment thereof is bound or covalently linked to a surface, such as a bead or plate (i.e., in an immobilized form). In one embodiment, the antibody is immobilized on a surface, such as Fc-coated wells. Alternatively, the antibody or fragment thereof is bound to the surface of a cell (e.g., immobilized on the surface of an antigen presenting cell (APC)). In another embodiment, the antibody is not immobilized on a surface when the composition is contacted with the antibody.

It will be appreciated that culturing the composition of γδ T cells is performed for a duration of time effective to produce an expanded population of γδ T cells. In one embodiment, a duration of time effective to produce an expanded population of γδ T cells is at least 7 days. Thus, in one embodiment, the composition of γδ T cells is cultured for at least 7 days. In a further embodiment, the composition is cultured for between 7 and 21 days, such as between 9 to 15 days. In yet further embodiments, the composition is cultured for about 10, 11, 12, 13 or 14 days. In some aspects, the composition is cultured for at least about 7 days. In some aspects, the composition is cultured for at least about 8 days. In some aspects, the composition is cultured for at least about 9 days. In some aspects, the composition is cultured for at least about 10 days. In some aspects, the composition is cultured for at least about 11 days. In some aspects, the composition is cultured for at least about 12 days. In some aspects, the composition is cultured for at least about 13 days. In some aspects, the composition is cultured for at least about 14 days.

In still further embodiments, the composition is cultured for at least 5 days, at least 6 days, at least 7 days, at least 8 days, at least 9 days, at least 10 days, at least 11 days, at least 12 days, at least 13 days, at least 14 days or at least 21 days, e.g. about 14 days or about 21 days to produce an expanded population of γδ T cells. In one embodiment, the composition is cultured for about 10, 11, 12, 13 or 14 days to produce an expanded population of γδ T cells. In some aspects, the composition is cultured for at least about 5 days to produce an expanded population of γδ T cells. In some aspects, the composition is cultured for at least about 6 days to produce an expanded population of γδ T cells. In some aspects, the composition is cultured for at least about 7 days to produce an expanded population of γδ T cells. In some aspects, the composition is cultured for at least about 8 days to produce an expanded population of γδ T cells. In some aspects, the composition is cultured for at least about 9 days to produce an expanded population of γδ T cells. In some aspects, the composition is cultured for at least about 10 days to produce an expanded population of γδ T cells. In some aspects, the composition is cultured for at least about 11 days to produce an expanded population of γδ T cells. In some aspects, the composition is cultured for at least about 12 days to produce an expanded population of γδ T cells. In some aspects, the composition is cultured for at least about 13 days to produce an expanded population of γδ T cells. In some aspects, the composition is cultured for at least about 14 days to produce an expanded population of γδ T cells. In some aspects, the composition is cultured for at least about 15 days to produce an expanded population of γδ T cells. In some aspects, the composition is cultured for at least about 16 days to produce an expanded population of γδ T cells. In some aspects, the composition is cultured for at least about 17 days to produce an expanded population of γδ T cells. In some aspects, the composition is cultured for at least about 18 days to produce an expanded population of γδ T cells. In some aspects, the composition is cultured for at least about 19 days to produce an expanded population of γδ T cells. In some aspects, the composition is cultured for at least about 20 days to produce an expanded population of γδ T cells. In some aspects, the composition is cultured for at least about 21 days to produce an expanded population of γδ T cells.

In certain embodiments of the present invention, the sample is cultured in media which is substantially free of serum (e.g. serum-free media or media containing a serum-replacement (SR)). Thus, in one embodiment, the sample is cultured in serum-free media. Such serum free medium may also include serum replacement medium, where the serum replacement is based on chemically defined components to avoid the use of human or animal derived serum. In an alternative embodiment, the sample is cultured in media which contains serum (e.g. human AB serum or fetal bovine serum (FBS)). In one embodiment, the sample is cultured in media which contains serum-replacement. In one embodiment, the sample is cultured in media which contains no animal-derived products. It will be appreciated that embodiments according to the invention wherein the sample is cultured in serum-free media have the advantage of avoiding issues with filtration, precipitation, contamination and supply of serum. Furthermore, animal derived products are not favoured for use in clinical grade manufacturing of human therapeutics.

In one embodiment, the methods as defined herein are performed in a vessel (e.g. an expansion vessel) comprising a gas permeable material. Such materials are permeable to gases such as oxygen, carbon dioxide and/or nitrogen to allow gaseous exchange between the contents of the vessel and the surrounding atmosphere. It will be appreciated that references herein to “vessel” include culture dishes, culture plates, single-well dishes, multi-well dishes, multi-well plates, flasks, multi-layer flasks, bottles (such as roller bottles), bioreactors, bags, tubes and the like. Such vessels are known in the art for use in methods involving expansion of non-adherent cells and other lymphocytes. However, vessels comprising a gas permeable material also surprisingly find utility in the isolation and expansion of γδ T cells which are considered as usually being adherent. The use of such vessels for culturing was found to greatly increase the yield of expanded γδ T cells. Such vessels were also found to preferentially support γδ T cells and other lymphocytes over fibroblasts and other stromal cells (e.g. epithelial cells), including adherent cell-types. See, e.g., Int'l Publication Nos. WO2020095058 and WO2020095059, each of which is incorporated by reference herein in its entirety. Thus, in one embodiment, the vessels comprising a gas permeable material as defined herein preferentially support γδ T cells and other lymphocytes (e.g. αβ T cells and/or NK cells). In a further embodiment, fibroblasts and/or other stromal cells (e.g. epithelial cells) are absent from cultures performed in vessels comprising a gas permeable material.

Such vessels comprising gas permeable materials may additionally comprise a gas permeable material that is non-porous. Thus, in one embodiment, the gas permeable material in non-porous. In some embodiments, the gas permeable material is a membrane film such as silicone, fluoroethylene polypropylene, polyolefin, or ethylene vinyl acetate copolymer. Furthermore, such vessels may comprise only a portion of gas permeable material, gas permeable membrane film or non-porous gas permeable material. Thus, according to a yet further embodiment, the vessel includes a top, a bottom and at least one sidewall, wherein at least part of the said vessel bottom comprises a gas permeable material that is in a substantially horizontal plane when said top is above said bottom. In one embodiment, the vessel includes a top, a bottom, and at least one sidewall, wherein at least a part of said bottom comprises the gas permeable material that is in a horizontal plane when said top is above said bottom. In a further embodiment, the vessel includes a top, a bottom and at least one sidewall, wherein the said at least one sidewall comprises a gas permeable material which may be in a vertical plane when said top is above said bottom, or may be a horizonal plane when said top is not above said bottom. It will be appreciated that in such embodiments, only a portion of said bottom or said side wall may comprise a gas permeable material. Alternatively, the entire of said bottom or entire of said sidewall may comprise a gas permeable material. In a yet further embodiment, said top of said vessel comprising a gas permeable material may be sealed, for example by utilisation of an O-ring. Such embodiments will be appreciated to prevent spillage or reduce evaporation of the vessel contents. Thus, in certain embodiments, the vessel comprises a liquid sealed container comprising a gas permeable material to allow gas exchange. In alternative embodiments, said top of said vessel comprising a gas permeable material is in the horizonal plane and above said bottom and is not sealed. Thus, in certain embodiments, said top is configured to allow gas exchange from the top of the vessel. In further embodiments, said bottom of the gas permeable container is configured to allow gas exchange from the bottom of the vessel. In a yet further embodiment, said vessel comprising a gas permeable material may be a liquid sealed container and further comprise inlet and outlet ports or tubes. Thus, in certain embodiments, the vessel comprising a gas permeable material includes a top, a bottom and optionally at least one sidewall, wherein at least a part of said top and said bottom comprise a gas permeable material and, if present, at least part of the at least one sidewall comprises a gas permeable material. Example vessels are described in WO2005035728 and U.S. Pat. No. 9,255,243 which are herein incorporated by reference. These vessels are also commercially available, such as the G-REX® cell culture devices provided by Wilson Wolf Manufacturing, such as the G-REX6 well-plate, G-REX24 well-plate and the G-REX10 vessel.

Suitably expanding the population of γδ T cells provides at least a 5-fold, especially at least a 10-fold or at least 15-fold, in particular at least a 20-fold number of γδ T cells. In some aspects, the fold change is relative to the starting population of γδ T cells.

The composition is cultured in media comprising IL-15. As used herein, “IL-15” refers to native or recombinant IL-15 or a variant thereof that acts as an agonist for one or more IL-15 receptor (IL-15R) subunits (e.g. mutants, muteins, analogues, subunits, receptor complexes, fragments, isoforms, and peptidomimetics thereof). IL-15, like IL-2, is a known T-cell growth factor that can support proliferation of an IL-2-dependent cell line, CTLL-2. IL-15 was first reported by Grabstein, et al. (Grabstein, et al. Science 1994. 264.5161:965-969) as a 114-amino acid mature protein. The term “IL-15,” as used herein, means native or recombinant IL-15 and muteins, analogs, subunits thereof, or complexes thereof (e.g. receptor complexes, e.g. sushi peptides, as described in WO 2007/046006), and each of which can stimulate proliferation of CTLL-2 cells. In the CTLL-2 proliferation assays, supernatants of cells transfected with recombinantly expressed precursor and in-frame fusions of mature forms of IL-15 can induce CTLL-2 cell proliferation.

Human IL-15 can be obtained according to the procedures described by Grabstein, et al. or by conventional procedures such as polymerase chain reaction (PCR). A deposit of human IL-15 cDNA was made with the ATCC® on Feb. 19, 1993 and assigned accession number 69245.

The amino acid sequence of human IL-15 (Gene ID 3600) is found in Genbank under accession locator NP000576.1 GI: 10835153 (isoform 1) and NP_751915.1 GI: 26787986 (isoform 2). The murine (Mus musculus) IL-15 amino acid sequence (Gene ID 16168) is found in Genbank under accession locator NP_001241676.1 GI: 363000984.

IL-15 can also refer to IL-15 derived from a variety of mammalian species, including, for example, human, simian, bovine, porcine, equine, and murine. An IL-15 “mutein” or “variant”, as referred to herein, is a polypeptide substantially homologous to a sequence of a native mammalian IL-15 but that has an amino acid sequence different from a native mammalian IL-15 polypeptide because of an amino acid deletion, insertion or substitution. Variants may comprise conservatively substituted sequences, meaning that a given amino acid residue is replaced by a residue having similar physiochemical characteristics. Examples of conservative substitutions include substitution of one aliphatic residue for another, such as Ile, Val, Leu, or Ala for one another, or substitutions of one polar residue for another, such as between Lys and Arg; Glu and Asp; or Gln and Asn. Other such conservative substitutions, for example, substitutions of entire regions having similar hydrophobicity characteristics, are well known. Naturally occurring IL-15 variants are also encompassed by the invention. Examples of such variants are proteins that result from alternate mRNA splicing events or from proteolytic cleavage of the IL-15 protein, wherein the IL-15 binding property is retained. Alternate splicing of mRNA may yield a truncated but biologically active IL-15 protein. Variations attributable to proteolysis include, for example, differences in the N- or C-termini upon expression in different types of host cells, due to proteolytic removal of one or more terminal amino acids from the IL-15 protein (generally from 1-10 amino acids). In some embodiments, the terminus of the protein can be modified to alter its physical properties, for example, with a chemical group such as polyethylene glycol (Yang, et al. Cancer 1995. 76:687-694). In some embodiments, the terminus or interior of the protein can be modified with additional amino acids (Clark-Lewis, et al. PNAS 1993. 90:3574-3577).

In some embodiments, the methods defined herein include IL-15 typically at a concentration of at least 0.1 ng/mL, such as at least 10 ng/ml (e.g. from 0.1 ng/ml to 10,000 ng/mL, from 1.0 ng/ml to 1,000 ng/mL, from 5 ng/ml to 800 ng/ml, from 10 ng/mL to 750 ng/mL, from 20 ng/mL to 500 ng/mL, from 50 ng/ml to 400 ng/ml, or from 100 ng/ml to 250 ng/ml, e.g. from 0.1 ng/ml to 1.0 ng/mL, from 1.0 ng/ml to 5.0 ng/mL, from 5.0 ng/mL to 10 ng/ml, from 10 ng/ml to 20 ng/mL, from 20 ng/ml to 100 ng/mL, from 20 ng/ml to 50 ng/mL, from 40 ng/ml to 70 ng/ml, from 50 ng/ml to 100 ng/mL, from 50 ng/ml to 60 ng/mL, from 100 ng/ml to 200 ng/mL, from 200 ng/mL to 500 ng/ml, or from 500 ng/mL to 1,000 ng/ml). In further embodiments, the methods defined herein include IL-15 typically at a concentration of less than 500 ng/ml, such as less than 250 ng/ml. In some embodiments, the concentration of IL-15 is about 100 ng/mL. In some embodiments defined herein, the IL-15 is included at a concentration from 5 ng/mL-300 ng/mL (e.g., 5 ng/mL-150 ng/ml) (e.g., 10 ng/ml-150 ng/mL) (e.g., 10 ng/mL-100 ng/ml). In some aspects, the IL-15 is included at a concentration from about 5 ng/mL-250 ng/mL, about 5 ng/ml-200 ng/ml, about 5 ng/ml-150 ng/ml, about 10 ng/ml-250 ng/ml, about 10 ng/ml-200 ng/mL, about 10 ng/ml-150 ng/mL, about 20 ng/mL-250 ng/ml, about 20 ng/ml-200 ng/mL, about 20 ng/mL-150 ng/mL, about 30 ng/ml-250 ng/mL, about 30 ng/ml-200 ng/ml, about 30 ng/mL-150 ng/mL, about 40 ng/ml-250 ng/mL, about 40 ng/ml-200 ng/ml, about 40 ng/mL-150 ng/mL, about 50 ng/ml-250 ng/ml, about 50 ng/mL-200 ng/ml, about 50 ng/mL-150 ng/ml, about 10 ng/mL-125 ng/ml, about 10 ng/ml-100 ng/ml, or about 20 ng/ml-100 ng/mL. In some aspects, the IL-15 is included at a concentration from about 5 ng/mL to about 150 ng/mL. In some aspects, the IL-15 is included at a concentration from about 5 ng/ml to about 125 ng/mL. In some aspects, the IL-15 is included at a concentration from about 5 ng/ml to about 100 ng/ml. In some aspects, the IL-15 is included at a concentration from about 10 ng/ml to about 150 ng/mL. In some aspects, the IL-15 is included at a concentration from about 10 ng/ml to about 125 ng/mL. In some aspects, the IL-15 is included at a concentration from about 10 ng/mL to about 100 ng/mL. In some aspects, the IL-15 is included at a concentration from about 15 ng/ml to about 150 ng/ml. In some aspects, the IL-15 is included at a concentration from about 5-300 (e.g., wherein the range is from 10-150). In some aspects, the IL-15 is included at a concentration from about 15 ng/ml to about 125 ng/ml. In some aspects, the IL-15 is included at a concentration from about 15 ng/ml to about 100 ng/mL. In some aspects, the IL-15 is included at a concentration from about 20 ng/ml to about 150 ng/mL. In some aspects, the IL-15 is included at a concentration from about 20 ng/ml to about 125 ng/mL. In some aspects, the IL-15 is included at a concentration from about 20 ng/ml to about 100 ng/mL. In some aspects, the IL-15 is included at a concentration from about 25 ng/ml to about 150 ng/mL. In some aspects, the IL-15 is included at a concentration from about 25 ng/mL to about 125 ng/mL. In some aspects, the IL-15 is included at a concentration from about 25 ng/ml to about 100 ng/mL.

In some aspects, the IL-15 is included at a concentration of about 5 ng/ml. In some aspects, the IL-15 is included at a concentration of about 10 ng/ml. In some aspects, the IL-15 is included at a concentration of about 15 ng/mL. In some aspects, the IL-15 is included at a concentration of about 20 ng/ml. In some aspects, the IL-15 is included at a concentration of about 21 ng/ml. In some aspects, the IL-15 is included at a concentration of about 25 ng/mL. In some aspects, the IL-15 is included at a concentration of about 30 ng/ml. In some aspects, the IL-15 is included at a concentration of about 35 ng/mL. In some aspects, the IL-15 is included at a concentration of about 40 ng/mL. In some aspects, the IL-15 is included at a concentration of about 45 ng/mL. In some aspects, the IL-15 is included at a concentration of about 50 ng/mL. In some aspects, the IL-15 is included at a concentration of about 60 ng/mL. In some aspects, the IL-15 is included at a concentration of about 70 ng/mL. In some aspects, the IL-15 is included at a concentration of about 80 ng/mL. In some aspects, the IL-15 is included at a concentration of about 90 ng/mL. In some aspects, the IL-15 is included at a concentration of about 100 ng/mL. In some aspects, the IL-15 is included at a concentration of about 110 ng/mL. In some aspects, the IL-15 is included at a concentration of about 120 ng/mL. In some aspects, the IL-15 is included at a concentration of about 130 ng/mL. In some aspects, the IL-15 is included at a concentration of about 140 ng/mL. In some aspects, the IL-15 is included at a concentration of about 150 ng/mL.

In one embodiment, the composition is cultured in media consisting essentially of an anti-CD3 antibody or fragment thereof, and IL-15. “Consisting essentially of” when used to define compositions and methods, shall mean excluding other elements of any essential significance to the composition for the intended purpose. Thus, a composition consisting essentially of the elements as defined herein would not exclude standard media components, e.g. culture medium (CTS OpTmizer, Thermo Fisher), plasma, serum and essential amino acids, such as L-glutamine (e.g. Glutamax).

Alternatively, the method (in particular, such as in step (ii)) may comprise culturing the composition enriched for γδ T cells in the presence of one or more additional cytokines selected from: IL-2, IL-21 and IL-1β.

In some aspects, the cells are seeded in a medium that comprises an anti-CD3 antibody and IL-15, wherein the medium does not comprise an additional exogenous cytokine. In some aspects, the cells are seeded in a medium that comprises an anti-CD3 antibody and IL-15, wherein the medium does not comprise IL-4. In some aspects, the cells are seeded in a medium that comprises an anti-CD3 antibody and IL-15, wherein the medium does not comprise IL-1β. In some aspects, the cells are seeded in a medium that comprises an anti-CD3 antibody and IL-15, wherein the medium does not comprise IL-21. In some aspects, the cells are seeded in a medium that comprises an anti-CD3 antibody and IL-15, wherein the medium does not comprise IFNγ. In some aspects, the cells are seeded in a medium that comprises an anti-CD3 antibody and IL-15, wherein the medium does not comprise IL-2. In some aspects, the cells are seeded in a medium that comprises an anti-CD3 antibody and IL-15, wherein the medium does not comprise IL-9.

In some aspects, hematopoietic-derived γδ cells are seeded in a medium that comprises an anti-CD3 antibody and IL-15, wherein the medium does not comprise an additional exogenous cytokine. In some aspects, hematopoietic-derived γδ cells are seeded in a medium that comprises an anti-CD3 antibody and IL-15, wherein the medium does not comprise IL-4. In some aspects, hematopoietic-derived γδ cells are seeded in a medium that comprises an anti-CD3 antibody and IL-15, wherein the medium does not comprise IL-1β. In some aspects, hematopoietic-derived γδ cells are seeded in a medium that comprises an anti-CD3 antibody and IL-15, wherein the medium does not comprise IL-21. In some aspects, hematopoietic-derived γδ cells are seeded in a medium that comprises an anti-CD3 antibody and IL-15, wherein the medium does not comprise IFNγ. In some aspects, hematopoietic-derived γδ cells are seeded in a medium that comprises an anti-CD3 antibody and IL-15, wherein the medium does not comprise IL-2. In some aspects, hematopoietic-derived γδ cells are seeded in a medium that comprises an anti-CD3 antibody and IL-15, wherein the medium does not comprise IL-9.

In some aspects, the additional IL-15 is added to the cell culture medium following seeding. In some aspects, the additional IL-15 is added on at least one day, on at least two days, on at least three days, on at least four days, on at least five days, on at least 6 days, or on at least 7 days (e.g., wherein the IL-15 is added in an amount from 5-150 ng/ml) (e.g., from 15-115 ng/ml). In some aspects, additional IL-15 is added on day 1 medium following seeding (e.g., wherein the IL-15 is added in an amount from 5-150 ng/ml) (e.g., from 15-115 ng/ml). In some aspects, additional IL-15 is added on day 2 medium following seeding (e.g., wherein the IL-15 is added in an amount from 5-150 ng/ml) (e.g., from 15-115 ng/ml). In some aspects, additional IL-15 is added on day 3 medium following seeding (e.g., wherein the IL-15 is added in an amount from 5-150 ng/ml) (e.g., from 15-115 ng/ml). In some aspects, additional IL-15 is added on day 4 medium following seeding (e.g., wherein the IL-15 is added in an amount from 5-150 ng/ml) (e.g., from 15-115 ng/ml). In some aspects, additional IL-15 is added on day 5 medium following seeding (e.g., wherein the IL-15 is added in an amount from 5-150 ng/ml) (e.g., from 15-115 ng/ml). In some aspects, additional IL-15 is added on day 6 medium following seeding (e.g., wherein the IL-15 is added in an amount from 5-150 ng/ml) (e.g., from 15-115 ng/ml). In some aspects, additional IL-15 is added on day 7 medium following seeding (e.g., wherein the IL-15 is added in an amount from 5-150 ng/ml) (e.g., from 15-115 ng/ml). In some aspects, additional IL-15 is added on day 8 medium following seeding (e.g., wherein the IL-15 is added in an amount from 5-150 ng/ml) (e.g., from 15-115 ng/ml). In some aspects, additional IL-15 is added on day 9 medium following seeding (e.g., wherein the IL-15 is added in an amount from 5-150 ng/ml) (e.g., from 15-115 ng/ml). In some aspects, additional IL-15 is added on day 10 medium following seeding (e.g., wherein the IL-15 is added in an amount from 5-150 ng/ml) (e.g., from 15-115 ng/ml). In some aspects, additional IL-15 is added on day 11 medium following seeding (e.g., wherein the IL-15 is added in an amount from 5-150 ng/ml) (e.g., from 15-115 ng/ml). In some aspects, additional IL-15 is added on day 12 medium following seeding (e.g., wherein the IL-15 is added in an amount from 5-150 ng/ml) (e.g., from 15-115 ng/ml). In some aspects, additional IL-15 is added on day 13 medium following seeding (e.g., wherein the IL-15 is added in an amount from 5-150 ng/ml) (e.g., from 15-115 ng/ml). In some aspects, additional IL-15 is added on day 14 following seeding (e.g., wherein the IL-15 is added in an amount from 5-150 ng/ml) (e.g., from 15-115 ng/ml).

In some aspects, additional IL-15 is added on days 4, 5, 6, and 7 following seeding (e.g., wherein the IL-15 is added in an amount from 5-150 ng/ml) (e.g., from 15-115 ng/ml). In some aspects, additional IL-15 is added on days 10, 11, and 12 following seeding (e.g., wherein the IL-15 is added in an amount from 5-150 ng/ml) (e.g., from 15-115 ng/ml). In some aspects, additional IL-15 is added on days 16, 17, and 18 following seeding (e.g., wherein the IL-15 is added in an amount from 5-150 ng/ml) (e.g., from 15-115 ng/ml).

In some aspects, no other cytokine is added with the additional IL-15. As such, in some aspects, the cells are cultured for the duration of the culture in the absence of exogenously added IL-4, IL-1β, IL-21, IFNγ, or any combination thereof.

In some aspects, the cells are cultured according to the methods disclosed herein for at least about 7 days, at least about 8 days, at least about 9 days, at least about 10 days, at least about 11 days, at least about 12 days, at least about 13 days, at least about 14 days, at least about 15 days, at least about 16 days, at least about 17 days, at least about 18 days, at least about 19 days, at least about 20 days, or at least about 21 days. In some aspects, the cells are cultured according to the methods disclosed herein for at least about 7 days. In some aspects, the cells are cultured according to the methods disclosed herein for at least about 8 days. In some aspects, the cells are cultured according to the methods disclosed herein for at least about 9 days. In some aspects, the cells are cultured according to the methods disclosed herein for at least about 10 days. In some aspects, the cells are cultured according to the methods disclosed herein for at least about 11 days. In some aspects, the cells are cultured according to the methods disclosed herein for at least about 12 days. In some aspects, the cells are cultured according to the methods disclosed herein for at least about 13 days. In some aspects, the cells are cultured according to the methods disclosed herein for at least about 14 days. In some aspects, the cells are cultured according to the methods disclosed herein for at least about 15 days. In some aspects, the cells are cultured according to the methods disclosed herein for at least about 16 days. In some aspects, the cells are cultured according to the methods disclosed herein for at least about 17 days. In some aspects, the cells are cultured according to the methods disclosed herein for at least about 18 days. In some aspects, the cells are cultured according to the methods disclosed herein for at least about 19 days. In some aspects, the cells are cultured according to the methods disclosed herein for at least about 20 days. In some aspects, the cells are cultured according to the methods disclosed herein for at least about 21 days.

In some aspects, the cells are contacted with IL-21. As used herein, “IL-21” refers to native or recombinant IL-21 or a variant thereof that acts as an agonist for one or more IL-21 receptor (IL-21R) subunits (e.g. mutants, muteins, analogues, subunits, receptor complexes, fragments, isoforms, and peptidomimetics thereof). Such agents can support proliferation of natural killer (NK) and cytotoxic (CD8⁺) T cells. Mature human IL-21 occurs as a 133 amino acid sequence (less the signal peptide, consisting of an additional 22 N-terminal amino acids). An IL-21 mutein is a polypeptide wherein specific substitutions to the Interleukin-21 protein have been made while retaining the ability to bind IL-21Rα, such as those described in U.S. Pat. No. 9,388,241. The IL-21 muteins can be characterized by amino acid insertions, deletions, substitutions and modifications at one or more sites in or at the other residues of the native IL-21 polypeptide chain. In accordance with this disclosure any such insertions, deletions, substitutions and modifications result in an IL-21 mutein that retains the IL-21R binding activity. Exemplary muteins can include substitutions of 1, 2, 3, 4, 5, 6, 7, 8, 9, 10 or more amino acids.

Nucleic acid encoding human IL-21 can be obtained by conventional procedures such as polymerase chain reaction (PCR). The amino acid sequence of human IL-21 (Gene ID 59067) is found in Genbank under accession locator NC_000004.12. The murine (Mus musculus) IL-21 amino acid sequence (Gene ID 60505) is found in Genbank under accession locator NC 000069.6.

IL-21 can also refer to IL-21 derived from a variety of mammalian species, including, for example, human, simian, bovine, porcine, equine, and murine. Variants may comprise conservatively substituted sequences, meaning that a given amino acid residue is replaced by a residue having similar physiochemical characteristics. Examples of conservative substitutions include substitution of one aliphatic residue for another, such as Ile, Val, Leu, or Ala for one another, or substitutions of one polar residue for another, such as between Lys and Arg; Glu and Asp; or Gln and Asn. Other such conservative substitutions, for example, substitutions of entire regions having similar hydrophobicity characteristics, are well known. Naturally occurring IL-21 variants are also encompassed by the invention. Examples of such variants are proteins that result from alternate mRNA splicing events or from proteolytic cleavage of the IL-21 protein, wherein the IL-21 binding property is retained. Alternate splicing of mRNA may yield a truncated but biologically active IL-21 protein. Variations attributable to proteolysis include, for example, differences in the N- or C-termini upon expression in different types of host cells, due to proteolytic removal of one or more terminal amino acids from the IL-21 protein (generally from 1-10 amino acids). In some embodiments, the terminus of the protein can be modified to alter its physical properties, for example, with a chemical group such as polyethylene glycol (Yang, et al. Cancer 1995. 76:687-694). In some embodiments, the terminus or interior of the protein can be modified with additional amino acids (Clark-Lewis, et al. PNAS 1993. 90:3574-3577).

In further embodiments, the methods defined herein include IL-21 typically at a concentration of at least 0.1 ng/ml, such as at least 1.0 ng/ml (e.g. from 0.1 ng/ml to 1,000 ng/mL, from 1.0 ng/ml to 100 ng/mL, from 1.0 ng/ml to 50 ng/mL, from 2 ng/ml to 50 ng/mL, from 3 ng/mL to 10 ng/ml, from 4 ng/mL to 8 ng/mL, from 5 ng/ml to 10 ng/ml, from 6 ng/ml to 8 ng/ml, e.g. from 0.1 ng/ml to 10 ng/ml, from 1.0 ng/ml to 5 ng/mL, from 1.0 ng/ml to 10 ng/mL, from 1.0 ng/mL to 20 ng/ml). In further embodiments, the methods defined herein include IL-21 typically at a concentration of less than 100 ng/mL, such as less than 50 ng/mL. In some aspects, the concentration of IL-21 is from 3 ng/mL to 40 ng/ml, from 4 ng/ml to 20 ng/mL, from 5 ng/mL to 15 ng/mL, or from 6 ng/mL to 10 ng/mL, In some aspects, the cells are not contacted with IL-21.

In some aspects, the cells are contacted with IL-2. As used herein, “IL-2” refers to native or recombinant IL-2 or a variant thereof that acts as an agonist for one or more IL-2 receptor (IL-2R) subunits (e.g. mutants, muteins, analogues, subunits, receptor complexes, fragments, isoforms, and peptidomimetics thereof). Such agents can support proliferation of an IL-2-dependent cell line, CTLL-2 (33; American Type Culture Collection (ATCC®) TIB 214). Mature human IL-2 occurs as a 133 amino acid sequence (less the signal peptide, consisting of an additional 20 N-terminal amino acids), as described in Fujita, et al. Cell 1986. 46.3:401-407. An IL-2 mutein is a polypeptide wherein specific substitutions to the Interleukin-2 protein have been made while retaining the ability to bind IL-2Rβ, such as those described in US 2014/0046026. The IL-2 muteins can be characterized by amino acid insertions, deletions, substitutions and modifications at one or more sites in or at the other residues of the native IL-2 polypeptide chain. In accordance with this disclosure any such insertions, deletions, substitutions and modifications result in an IL-2 mutein that retains the IL-2Rβ binding activity. Exemplary muteins can include substitutions of 1, 2, 3, 4, 5, 6, 7, 8, 9, 10 or more amino acids.

Nucleic acid encoding human IL-2 can be obtained by conventional procedures such as polymerase chain reaction (PCR). The amino acid sequence of human IL-2 (Gene ID 3558) is found in Genbank under accession locator NP_000577.2 GI: 28178861. The murine (Mus musculus) IL-2 amino acid sequence (Gene ID 16183) is found in Genbank under accession locator NP_032392.1 GI: 7110653.

IL-2 can also refer to IL-2 derived from a variety of mammalian species, including, for example, human, simian, bovine, porcine, equine, and murine. Variants may comprise conservatively substituted sequences, meaning that a given amino acid residue is replaced by a residue having similar physiochemical characteristics. Examples of conservative substitutions include substitution of one aliphatic residue for another, such as Ile, Val, Leu, or Ala for one another, or substitutions of one polar residue for another, such as between Lys and Arg; Glu and Asp; or Gln and Asn. Other such conservative substitutions, for example, substitutions of entire regions having similar hydrophobicity characteristics, are well known. Naturally occurring IL-2 variants are also encompassed by the invention. Examples of such variants are proteins that result from alternate mRNA splicing events or from proteolytic cleavage of the IL-2 protein, wherein the IL-2 binding property is retained. Alternate splicing of mRNA may yield a truncated but biologically active IL-2 protein. Variations attributable to proteolysis include, for example, differences in the N- or C-termini upon expression in different types of host cells, due to proteolytic removal of one or more terminal amino acids from the IL-2 protein (generally from 1-10 amino acids). In some embodiments, the terminus or interior of the protein can be modified to alter its physical properties, for example, with a chemical group such as polyethylene glycol (Yang, et al. Cancer 1995. 76:687-694). In some embodiments, the terminus or interior of the protein can be modified with additional amino acids (Clark-Lewis, et al. PNAS 1993. 90:3574-3577).

In certain embodiments, the methods defined herein include IL-2 typically at a concentration of at least 10 IU/mL, such as at least 100 IU/mL (e.g. from 10 IU/mL to 1,000 IU/mL, from 20 IU/mL to 800 IU/mL, from 25 IU/mL to 750 IU/mL, from 30 IU/mL to 700 IU/mL, from 40 IU/mL to 600 IU/mL, from 50 IU/mL to 500 IU/mL, from 75 IU/mL to 250 IU/mL, or from 100 IU/mL to 200 IU/mL, e.g. from 10 IU/mL to 20 IU/mL, from 20 IU/mL to 30 IU/mL, from 30 IU/mL to 40 IU/mL, from 40 IU/mL to 50 IU/mL, from 50 IU/mL to 75 IU/mL, from 75 IU/mL to 100 IU/mL, from 100 IU/mL to 150 IU/mL, from 150 IU/mL to 200 IU/mL, from 200 IU/mL to 500 IU/mL, or from 500 IU/mL to 1,000 IU/mL). In certain embodiments, the methods defined herein include IL-2 typically at a concentration of less than 1,000 IU/mL, such as less than 500 IU/mL. In some embodiments, the concentration of IL-2 is about 100 IU/mL. In some aspects, the cells are not contacted with IL-2.

In some aspects, the cells are contacted with IL-1β. As used herein, “IL-1β” refers to native or recombinant IL-1β or a variant thereof that acts as an agonist for one or more IL-1 receptor (IL-1R) subunits (e.g. mutants, muteins, analogues, subunits, receptor complexes, fragments, isoforms, and peptidomimetics thereof). IL-1 is a pro-inflammatory cytokine that plays a major role in a wide range of diseases, including inflammatory diseases. It consists of two molecular species, IL-1α and IL-1β, which share only limited sequence identity but exert similar biological activities through binding to IL-1 receptor (type I and type II). Mature human IL-1β occurs as a 153 amino acid sequence after cleavage of 116 amino acids from the N-terminus in the precursor polypeptide by CASP1, as described in Andrei et al. (2004) PNAS 101 (26): 9745-9750. An IL-1β mutein is a polypeptide wherein specific substitutions to the IL-1β protein have been made while retaining the ability to bind IL-1R. The IL-1β muteins can be characterized by amino acid insertions, deletions, substitutions and modifications at one or more sites in or at the other residues of the native IL-1β polypeptide chain. In accordance with this disclosure any such insertions, deletions, substitutions and modifications result in an IL-1β mutein that retains the IL-1R binding activity. Exemplary muteins can include substitutions of 1, 2, 3, 4, 5, 6, 7, 8, 9, 10 or more amino acids.

Nucleic acid encoding human IL-1β can be obtained by conventional procedures such as polymerase chain reaction (PCR). The amino acid sequence of human IL-1β (Gene ID 3553) is found in Genbank under accession locator NP_000567 or in UniProt under accession number P01584. The murine (Mus musculus) IL-1β amino acid sequence (Gene ID 16176) is found in Genbank under accession locator NP_032387 or in UniProt under accession number P10749.

IL-1β can also refer to IL-1β derived from a variety of mammalian species, including, for example, human, simian, bovine, porcine, equine, and murine. Variants may comprise conservatively substituted sequences, meaning that a given amino acid residue is replaced by a residue having similar physiochemical characteristics. Examples of conservative substitutions include substitution of one aliphatic residue for another, such as Ile, Val, Leu, or Ala for one another, or substitutions of one polar residue for another, such as between Lys and Arg; Glu and Asp; or Gln and Asn. Other such conservative substitutions, for example, substitutions of entire regions having similar hydrophobicity characteristics, are well known. Naturally occurring IL-1β variants are also encompassed by the invention. Examples of such variants are proteins that result from alternate mRNA splicing events or from proteolytic cleavage of the IL-1β protein, wherein the IL-1β binding property is retained.

In certain embodiments, the methods defined herein include IL-1β typically at a concentration of at least 100 IU/mL, such as at least 1,000 IU/mL (e.g. from 100 IU/mL to 8,000 IU/mL, from 250 IU/mL to 7,000 IU/mL, from 500 IU/mL to 6,000 IU/mL, or from 1,000 IU/mL to 5,000 IU/mL). In certain embodiments, the methods defined herein include IL-1β typically at a concentration of less than 8,000 IU/mL, such as less than 5,000 IU/mL. In some embodiments, the concentration of IL-1β is about 4,500 IU/mL. In some aspects, the cells are not contacted with IL-1β.

In contrast to previous methods for expanding γδ T cells described in the art, the current method cultures the composition enriched for γδ T cells in the absence of IL-4. Therefore, methods of the invention are performed in media that does not contain (and is not supplemented) with IL-4.

In some aspects, the cells are not contacted with IL-4. As used herein, “IL-4” refers to native or recombinant IL-4 or a variant thereof that acts as an agonist for one or more IL-4 receptor (IL-4R) subunits (e.g. mutants, muteins, analogues, subunits, receptor complexes, fragments, isoforms, and peptidomimetics thereof). Such agents can support differentiation of naïve helper T cells (Th0 cells) to Th2 cells. Mature human IL-4 occurs as a 129 amino acid sequence (less the signal peptide, consisting of an additional 24 N-terminal amino acids). An IL-4 mutein is a polypeptide wherein specific substitutions to the Interleukin-4 protein have been made while retaining the ability to bind IL-4Rα, such as those described in U.S. Pat. No. 6,313,272. The IL-4 muteins can be characterized by amino acid insertions, deletions, substitutions and modifications at one or more sites in or at the other residues of the native IL-4 polypeptide chain. In accordance with this disclosure any such insertions, deletions, substitutions and modifications result in an IL-4 mutein that retains the IL-2Rα binding activity. Exemplary muteins can include substitutions of 1, 2, 3, 4, 5, 6, 7, 8, 9, 10 or more amino acids.

Nucleic acid encoding human IL-4 can be obtained by conventional procedures such as polymerase chain reaction (PCR). The amino acid sequence of human IL-4 (Gene ID 3565) is found in Genbank under accession locator NG 023252. The murine (Mus musculus) IL-4 amino acid sequence (Gene ID 16189) is found in Genbank under accession locator NC_000077.6.

IL-4 can also refer to IL-4 derived from a variety of mammalian species, including, for example, human, simian, bovine, porcine, equine, and murine. Variants may comprise conservatively substituted sequences, meaning that a given amino acid residue is replaced by a residue having similar physiochemical characteristics. Examples of conservative substitutions include substitution of one aliphatic residue for another, such as Ile, Val, Leu, or Ala for one another, or substitutions of one polar residue for another, such as between Lys and Arg; Glu and Asp; or Gln and Asn. Other such conservative substitutions, for example, substitutions of entire regions having similar hydrophobicity characteristics, are well known. Naturally occurring IL-4 variants are also encompassed by the invention. Examples of such variants are proteins that result from alternate mRNA splicing events or from proteolytic cleavage of the IL-4 protein, wherein the IL-4 binding property is retained. Alternate splicing of mRNA may yield a truncated but biologically active IL-4 protein. Variations attributable to proteolysis include, for example, differences in the N- or C-termini upon expression in different types of host cells, due to proteolytic removal of one or more terminal amino acids from the IL-4 protein (generally from 1-10 amino acids).

### Composition and Markers

In some embodiments, at least 80%, such as 85%, 90%, 95%, 97%, 98%, 99%, 99.5%, of the cells present in the cell population comprise NK cells and γδ T cells (e.g., Vδ1 T cells). In some embodiments, at least 80%, such as 85%, 90%, 95%, 97%, 98%, 99%, 99.5%, of the cells present in the cell population obtained by the methods described herein comprise NK cells and γδ T cells. In one embodiment, at least 85% of the cells present in the cell population comprise NK and γδ T cells. In a further embodiment, at least 90% of the cells present in the cell population comprise NK cells and γδ T cells. In a yet further embodiment, at least 95% of the cells present in the cell population comprise NK cells and γδ T cells.

In some embodiments, at least 50% of the cells present in the composition are γδ T cells. In some embodiments, the composition comprises at least about 50% γδ T cells, such as at least about 55%, 60%, 65%, 70%, 75%, 80%, 85%, 90% or 95% γδ T cells. In a further embodiment, the composition comprises at least about 60% γδ T cells, such as greater than about 70% γδ T cells. In other embodiments, at least 80%, such as at least 90%, of cells present in the composition are γδ T cells. In one embodiment, the composition comprises at least about 82% γδ T cells.

In some embodiments, the composition comprises at least about 30% Vδ1 T cells, such as at least about 31%, 32%, 33%, 34%, 35%, 36%, 37%, 38%, 39%, 40%, 41%, 42%, 43%, 44%, 45%, 46%, 47%, 48%, 49% or 50% Vδ1 T cells. In a further embodiment, the composition comprises at least about 40% Vδ1 T cells, such as at least about 50% Vδ1 T cells. In some embodiments, the composition comprises between about 50% to 70% Vδ1 T cells. In one embodiment, the composition comprises about 60% Vδ1 T cells.

In some aspects, the composition comprises less than about 1% αβ T cells. In some aspects, the composition comprises less than about 1% αβ T cells, less than about 0.9% αβ T cells, less than about 0.8% αβ T cells, less than about 0.7% αβ T cells, less than about 0.6% αβ T cells, less than about 0.5% αβ T cells, less than about 0.4% αβ T cells, less than about 0.3% αβ T cells, less than about 0.2% αβ T cells, less than about 0.1% αβ T cells, less than about 0.09% αβ T cells, less than about 0.08% αβ T cells, less than about 0.07% αβ T cells, less than about 0.06% αβ T cells, less than about 0.05% αβ T cells, less than about 0.04% αβ T cells, less than about 0.05% αβ T cells, less than about 0.04% αβ T cells, less than about 0.03% αβ T cells, less than about 0.02% αβ T cells, less than about 0.01% αβ T cells, less than about 0.009% αβ T cells, less than about 0.008% αβ T cells, less than about 0.007% αβ T cells, less than about 0.006% αβ T cells, less than about 0.005% αβ T cells. In some aspects, the composition comprises less than about 0.9% αβ T cells. In some aspects, the composition comprises less than about 0.8% αβ T cells. In some aspects, the composition comprises less than about 0.7% αβ T cells. In some aspects, the composition comprises less than about 0.6% αβ T cells. In some aspects, the composition comprises less than about 0.5% αβ T cells. In some aspects, the composition comprises less than about 0.4% αβ T cells. In some aspects, the composition comprises less than about 0.3% αβ T cells. In some aspects, the composition comprises less than about 0.2% αβ T cells. In some aspects, the composition comprises less than about 0.1% αβ T cells. In some aspects, the composition comprises less than about 0.09% αβ T cells. In some aspects, the composition comprises less than about 0.08% αβ T cells. In some aspects, the composition comprises less than about 0.07% αβ T cells. In some aspects, the composition comprises less than about 0.06% αβ T cells. In some aspects, the composition comprises less than about 0.05% αβ T cells. In some aspects, the composition comprises less than about 0.04% αβ T cells. In some aspects, the composition comprises less than about 0.03% αβ T cells. In some aspects, the composition comprises less than about 0.02% αβ T cells. In some aspects, the composition comprises less than about 0.01% αβ T cells. In some aspects, the composition comprises a concentration of αβ T cells that is below detectable limits.

In some aspects, at least about 50% of cells isolated prior to the expansion methods disclosed herein are αβ T cells, and less than about 1% of the cells are αβ T cells in the composition following expansion according to the methods disclosed herein. In some aspects, at least about 45% of cells isolated prior to the expansion methods disclosed herein are αβ T cells, and less than about 1% of the cells are αβ T cells in the composition following expansion according to the methods disclosed herein. In some aspects, at least about 40% of cells isolated prior to the expansion methods disclosed herein are αβ T cells, and less than about 1% of the cells are αβ T cells in the composition following expansion according to the methods disclosed herein. In some aspects, at least about 35% of cells isolated prior to the expansion methods disclosed herein are αβ T cells, and less than about 1% of the cells are αβ T cells in the composition following expansion according to the methods disclosed herein. In some aspects, at least about 30% of cells isolated prior to the expansion methods disclosed herein are αβ T cells, and less than about 1% of the cells are αβ T cells in the composition following expansion according to the methods disclosed herein. In some aspects, at least about 25% of cells isolated prior to the expansion methods disclosed herein are αβ T cells, and less than about 1% of the cells are αβ T cells in the composition following expansion according to the methods disclosed herein. In some aspects, at least about 20% of cells isolated prior to the expansion methods disclosed herein are αβ T cells, and less than about 1% of the cells are αβ T cells in the composition following expansion according to the methods disclosed herein. In some aspects, at least about 15% of cells isolated prior to the expansion methods disclosed herein are αβ T cells, and less than about 1% of the cells are αβ T cells in the composition following expansion according to the methods disclosed herein. In some aspects, at least about 10% of cells isolated prior to the expansion methods disclosed herein are αβ T cells, and less than about 1% of the cells are αβ T cells in the composition following expansion according to the methods disclosed herein. In some aspects, at least about 5% of cells isolated prior to the expansion methods disclosed herein are αβ T cells, and less than about 1% of the cells are αβ T cells in the composition following expansion according to the methods disclosed herein.

In one embodiment, at least 50% of the expanded γδ T cells present in the cell population express CD56. In one embodiment, at least about 60%, such as at least 65%, 70%, 75% or 80% of the expanded γδ T cells present in the cell population express CD56. In a further embodiment, at least 80% of the expanded γδ T cells present in the cell population express CD56, for example after a period of about 14 days (such as 14 days) of culture.

CD56, also known as Neural Cell Adhesion Molecule (NCAM), is an adhesion molecule of the immunoglobulin (Ig) superfamily that correlates with high cytotoxicity in NK cells, αβ T cells and γδ T cells. It has been shown to participate in cis and trans binding to itself which contributes to lymphocyte activation. It has also been shown to be fundamental for immunological synapse formation between lymphocytes, lymphocytes and antigen presenting cells (APCs), as well as lymphocytes and target cells (Nussbaumer and Thurnher (2020) Cells 9 (3): 772). Cell phenotype can also be defined by the cell-surface density of CD56. There are therefore recognised sub-types known in the art of CD56^{bright} and CD56^{dim} cells. The increased expression and/or intensity of CD56 on γδ T cells have been shown to have a correlation with enhanced killing. CD56 surface expression can be determined using methods known in the art, such as through analysis of the staining intensity via flow cytometry. Dividing cells into CD56^{bright} and CD56^{dim} is understood in the art, for example as described in Van Acker et al. (2017) Front. Immunol. 8:892. Such methods compare the sample population to a reference population of known CD56 expression levels, such as a cell population containing NK cells. NK cells have distinct CD56 expression levels that can be identified as bright and dim, so by establishing gating strategies using this reference population, the sample population can also be sorted into CD56^{bright} and CD56^{dim} fractions.

In one embodiment, the cell population comprises γδ T cells that express CD56, NKp30, CD57, GITR, TIGIT, CCR6, CCR2, CCR5 and/or CXCR6, such as CD56, NKp30, CD57, GITR and/or TIGIT. In a further embodiment, the cell population comprises γδ T cells that express CD56, NKp30, CD57, GITR and TIGIT. In some aspects, the cell population comprises γδ T cells that express NKp30. In some aspects, the cell population comprises γδ T cells that express NKG2D. In some aspects, the cell population comprises γδ T cells that express DNAM-1. In some aspects, the cell population comprises γδ T cells that express CD57. In some aspects, the cell population comprises γδ T cells that express GITR. In some aspects, the cell population comprises γδ T cells that express TIGIT. In some aspects, the cell population comprises γδ T cells that express CXCR3. In some aspects, the cell population comprises γδ T cells that express CXCR4. In some aspects, the cell population comprises γδ T cells that express CCR4. In some aspects, the cell population comprises γδ T cells that express CCR6. In some aspects, the cell population comprises γδ T cells that express CCR2. In some aspects, the cell population comprises γδ T cells that express CCR5. In some aspects, the cell population comprises γδ T cells that express CXCR6.

In some aspects, the cell population comprises γδ T cells that secrete IFNδ. In some aspects, the cell population comprises γδ T cells that secrete IFNδ at higher levels than cells expanded using standard techniques. In some aspects, the cell population comprises γδ T cells that secrete GM-CSF. In some aspects, the cell population comprises γδ T cells that secrete GM-CSF at higher levels than cells expanded using standard techniques. In some aspects, the cell population comprises γδ T cells that secrete CXCL9. In some aspects, the cell population comprises γδ T cells that secrete CXCL9 at higher levels than cells expanded using standard techniques. In some aspects, the cell population comprises γδ T cells that secrete CXCL10. In some aspects, the cell population comprises γδ T cells that secrete CXCL10 at higher levels than cells expanded using standard techniques. In some aspects, the cell population comprises γδ T cells that secrete CCL3. In some aspects, the cell population comprises γδ T cells that secrete CCL3 at higher levels than cells expanded using standard techniques. In some aspects, the cell population comprises γδ T cells that secrete CCL4. In some aspects, the cell population comprises γδ T cells that secrete CCL4 at higher levels than cells expanded using standard techniques. In some aspects, the cell population comprises γδ T cells that secrete CCL7. In some aspects, the cell population comprises γδ T cells that secrete CCL7 at higher levels than cells expanded using standard techniques. In some aspects, the cell population comprises γδ T cells that secrete CCL20. In some aspects, the cell population comprises γδ T cells that secrete CCL20 at higher levels than cells expanded using standard techniques.

Methods of expansion as provided herein, in some embodiments, yield an expanded cell population of γδ T cells (in particular, Vδ1 T cells) having a high expression of particular markers, relative to a reference population (e.g. the isolated population of γδ T cells prior to the expansion step). Such markers may include, for example, CD56, NKp30, CD57, GITR and/or TIGIT. In some embodiments, the expanded cell population of γδ T cells (in particular, Vδ1 T cells) may have a frequency of CD56+ cells of more than about 20%, such as more than about 40%, more than about 50%, or more than about 60%. In some embodiments, more than about 60% of the cell population comprises γδ T cells (in particular, Vδ1 T cells) that express CD56. In some embodiments, the expanded cell population of γδ T cells (in particular, Vδ1 T cells) may have a frequency of NKp30+ cells of more than about 20%, such as more than about 25%, more than about 30%, or more than about 35%. In some embodiments, more than about 35% of the cell population comprises γδ T cells that express NKp30. In some embodiments, the expanded cell population of γδ T cells (in particular, Vδ1 T cells) may have a frequency of CD57+ cells of more than about 20%, such as more than about 30%, more than about 40%, or more than about 50%. In some embodiments, more than about 50% of the cell population comprises γδ T cells that express CD57. In some embodiments, the expanded cell population of γδ T cells (in particular, Vδ1 T cells) may have a frequency of GITR+ cells of more than about 20%, such as more than about 30%, more than about 40%, or more than about 50%. In some embodiments, more than about 50% of the cell population comprises γδ T cells that express GITR. In some embodiments, the expanded cell population of γδ T cells (in particular, Vδ1 T cells) may have a frequency of TIGIT+ cells of more than about 25%, such as more than about 30%, more than about 35%, more than about 40%, or more than about 45%. In some embodiments, more than about 45% of the cell population comprises γδ T cells that express GITR.

In one embodiment, the cell population comprises γδ T cells with a low (or undetectable) expression of LAG-3, PD-1 and CTLA-4. In some embodiments, less than about 15% of the cell population comprises γδ T cells that express LAG-3, PD-1 or CTLA-4 (i.e. >15% γδ T cells are LAG-3+, PD-1+ or CTLA-4+).

Methods of expansion as provided herein, in some embodiments, yield an expanded cell population of γδ T cells (in particular, Vδ1 T cells) having a low expression of particular markers, relative to a reference population (e.g. the isolated population of γδ T cells prior to the expansion step). Such markers may include, for example, CD27. In some embodiments, the expanded cell population of γδ T cells (in particular, Vδ1 T cells) may have a frequency of CD27+ cells of less than about 90%, such as less than about 85%, less than about 80%, or less than about 75%. In certain embodiments, the expanded cell population of γδ T cells (in particular, Vδ1 T cells) has a frequency of CD27+ cells of less than 75%. In some embodiments, less than about 75% of the cell population comprises γδ T cells that express CD27.

In one embodiment, the cell population comprises γδ T cells with a low (or undetectable) expression of LAG-3, PD-1 and CTLA-4. In some embodiments, less than about 15% of the cell population comprises γδ T cells that express LAG-3, PD-1 or CTLA-4 (i.e. has a frequency of LAG-3+, PD-1+ or CTLA-4+ cells of less than 15%).

In one embodiment, the cell population comprises γδ T cells with a low (or undetectable) expression of CD62L and CCR7. In some embodiments, less than about 15% of the cell population comprises γδ T cells that express CD62L or CCR7 (i.e. has a frequency of CD62L+ or CCR7+ cells of less than 15%).

In some aspects, the γδ T cell population (e.g., Vδ1 γδ T cell population) obtained by methods described herein has increased persistence of in vivo cytotoxicity as compared to similarly engineered cells obtained by standard methods. In some aspects, the γδ T cell population obtained by methods described herein are capable of in vivo cytotoxicity for at least about 7 days, at least about 8 days, at least about 9 days, at least about 10 days, at least about 11 days, at least about 12 days, at least about 13 days, at least about 14 days, at least about 15 days, at least about 16 days, at least about 17 days, at least about 18 days, at least about 19 days, at least about 20 days, or at least about 21 days. In some aspects, the γδ T cell population (e.g., Vδ1 γδ T cell population) obtained by methods described herein are capable of in vivo cytotoxicity for at least about 7 days. In some aspects, the γδ T cell population (e.g., Vδ1 γδ T cell population) obtained by methods described herein are capable of in vivo cytotoxicity for at least about 8 days. In some aspects, the γδ T cell population (e.g., Vδ1 γδ T cell population) obtained by methods described herein are capable of in vivo cytotoxicity for at least about 9 days. In some aspects, the γδ T cell population (e.g., Vδ1 γδ T cell population) obtained by methods described herein are capable of in vivo cytotoxicity for at least about 10 days. In some aspects, the γδ T cell population (e.g., Vδ1 γδ T cell population) obtained by methods described herein are capable of in vivo cytotoxicity for at least about 11 days. In some aspects, the γδ T cell population (e.g., Vδ1 γδ T cell population) obtained by methods described herein are capable of in vivo cytotoxicity for at least about 12 days. In some aspects, the γδ T cell population (e.g., Vδ1 γδ T cell population) obtained by methods described herein are capable of in vivo cytotoxicity for at least about 13 days. In some aspects, the γδ T cell population (e.g., Vδ1 γδ T cell population) obtained by methods described herein are capable of in vivo cytotoxicity for at least about 14 days. In some aspects, the γδ T cell population (e.g., Vδ1 γδ T cell population) obtained by methods described herein are capable of in vivo cytotoxicity for at least about 15 days. In some aspects, the γδ T cell population (e.g., Vδ1 γδ T cell population) obtained by methods described herein are capable of in vivo cytotoxicity for at least about 16 days. In some aspects, the γδ T cell population (e.g., Vδ1 γδ T cell population) obtained by methods described herein are capable of in vivo cytotoxicity for at least about 17 days. In some aspects, the γδ T cell population (e.g., Vδ1 γδ T cell population) obtained by methods described herein are capable of in vivo cytotoxicity for at least about 18 days. In some aspects, the γδ T cell population (e.g., Vδ1 γδ T cell population) obtained by methods described herein are capable of in vivo cytotoxicity for at least about 19 days. In some aspects, the γδ T cell population (e.g., Vδ1 γδ T cell population) obtained by methods described herein are capable of in vivo cytotoxicity for at least about 20 days. In some aspects, the γδ T cell population (e.g., Vδ1 γδ T cell population) obtained by methods described herein are capable of in vivo cytotoxicity for at least about 21 days.

In some aspects, the γδ T cell population (e.g., Vδ1 γδ T cell population) obtained by methods described herein are capable of in vivo cytotoxicity, despite having little proliferation. Though the efficacy of αβ T cell therapies are dependent on T cell proliferation, the methods disclosed herein generate γδ T cell capable of highly effective cytotoxicity despite having very minimal proliferation. In some aspects, the γδ T cell population (e.g., Vδ1 γδ T cell population) has a proliferation rate of less than about 15-fold, less than about 14-fold, less than about 13-fold, less than about 12-fold, less than about 11-fold, less than about 10-fold, less than about 9-fold, less than about 8-fold, less than about 7-fold, less than about 6-fold, less than about 5-fold, less than about 4-fold, less than about 3-fold, or less than about 2-fold. In some aspects, the γδ T cell population (e.g., Vδ1 γδ T cell population) obtained by methods described herein are capable of in vivo cytotoxicity for at least 7 days, wherein the γδ T cell population (e.g., Vδ1 γδ T cell population) has a proliferation rate of less than a 15-fold increase over the at least 7 days. In some aspects, the γδ T cell population (e.g., Vδ1 γδ T cell population) obtained by methods described herein are capable of in vivo cytotoxicity for at least 10 days, wherein the γδ T cell population (e.g., Vδ1 γδ T cell population) has a proliferation rate of less than a 15-fold increase over the at least 10 days. In some aspects, the γδ T cell population (e.g., Vδ1 γδ T cell population) obtained by methods described herein are capable of in vivo cytotoxicity for at least 14 days, wherein the γδ T cell population (e.g., Vδ1 γδ T cell population) has a proliferation rate of less than a 15-fold increase over the at least 14 days. In some aspects, the γδ T cell population (e.g., Vδ1 γδ T cell population) obtained by methods described herein are capable of in vivo cytotoxicity for at least 18 days, wherein the γδ T cell population (e.g., Vδ1 γδ T cell population) has a proliferation rate of less than a 15-fold increase over the at least 18 days. In some aspects, the γδ T cell population (e.g., Vδ1 γδ T cell population) obtained by methods described herein are capable of in vivo cytotoxicity for at least 21 days, wherein the γδ T cell population (e.g., Vδ1 γδ T cell population) has a proliferation rate of less than a 15-fold increase over the at least 21 days.

In some aspects, the in vivo cytotoxicity is against a cancer cell. In some aspects, the cancer cell is of a solid tumor. In some aspects, the cancer comprises bone cancer, pancreatic cancer, skin cancer, cancer of the head or neck, cutaneous or intraocular malignant melanoma, lung cancer (e.g., non-small cell lung cancer (NSCLC) or small cell lung cancer (SCLC)), uterine cancer, ovarian cancer, rectal cancer, cancer of the anal region, stomach cancer, testicular cancer, uterine cancer, carcinoma of the fallopian tubes, carcinoma of the endometrium, carcinoma of the cervix, carcinoma of the vagina, carcinoma of the vulva, Hodgkin's Disease, non-Hodgkin's lymphoma, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, chronic or acute leukemia, acute myeloid leukemia (AML) (e.g., relapsed or refractory AML), chronic myeloid leukemia, acute lymphoblastic leukemia, chronic lymphocytic leukemia (ALL), chronic myelogenous leukemia, solid tumors of childhood, lymphocytic lymphoma, cancer of the bladder, cancer of the kidney or ureter, carcinoma of the renal pelvis, neoplasm of the central nervous system (CNS), primary CNS lymphoma, tumor angiogenesis, spinal axis tumor, brain stem glioma, pituitary adenoma, Kaposi's sarcoma, epidermoid cancer, squamous cell cancer, T-cell lymphoma, environmentally induced cancers including those induced by asbestos, or any combination thereof. In some aspects, the cancer is locally advanced. In some aspects, the cancer is metastatic. In some aspects, the cancer is refractory. In some aspects, the cancer is relapsed. In some aspects, the cancer is refractory or relapsed following one or more prior anti-cancer therapy. In some aspects, the one or more prior anti-cancer therapies comprise a standard of care therapy.

### Engineered Cells

The γδ T cells described herein may also be gene engineered for enhanced therapeutic properties. For example, the cells may be engineered to express exogenous nucleic acids which encode cell surface receptors and/or secretory proteins.

One example of cell engineering includes CAR-T therapy. This may involve the generation of engineered receptors (such as chimeric antigen receptors or modified T cell receptors) to re-program the T cell with a new specificity, e.g. the specificity of a monoclonal antibody. The engineered receptor may make the T cells specific for malignant cells and therefore useful for cancer immunotherapy. For example, the T cells may recognise cancer cells expressing a tumour antigen, such as a tumour specific antigen that is not expressed by normal somatic cells from the subject tissue, a tumour associated antigen which is preferentially overexpressed on cancer cells compared to healthy somatic cells or antigens expressed in the context of stress events such as oxidative stress, DNA damage, UV radiation, EGF receptor stimulation; or other means for identifying cancerous versus noncancerous cells. Thus, the CAR-modified T cells may be used for adoptive T cell therapy of, for example, cancer patients.

According to a further aspect of the invention, there is provided a method for engineering γδ T cells, said method comprising the steps of:
(i) preparing a composition enriched for γδ T cells using the method as described herein;
(ii) transducing the composition with an exogenous nucleic acid for expression in the γδ T cells; and
(iii) culturing the transduced composition to expand the engineered γδ T cells.

It will be understood that transducing the composition with an exogenous nucleic acid for expression in the γδ T cells according to step (ii) thereby produces engineered γδ T cells expressing the exogenous nucleic acid. In some embodiments, the exogenous nucleic acid encodes a surface receptor. In a further embodiment, the surface receptor is a chimeric antigen receptor (CAR) recognizing a tumour antigen.

In one embodiment, the methods described herein comprise transducing the composition of γδ T cells to express a surface receptor of interest, such as a CAR recognizing a tumour antigen. Any such CAR may be used in the present invention, including CARs targeting CD19, Mesothelin (MSLN) or other known tumour associated antigens.

According to a further aspect of the invention, there is provided a method for engineering γδ T cells, said method comprising the steps of:
(i) preparing a composition enriched for γδ T cells according to the methods as described herein;
(ii) transducing the composition to express a chimeric antigen receptor (CAR); and
(iii) culturing the transduced composition to expand the engineered γδ T cells.

Thus, in some embodiments step (iii) comprises culturing the transduced composition in the presence of feeder cells.

In certain embodiments, the composition is transduced using a viral vector. Such viral vectors are known in the art and the skilled person will be able to recognise the appropriate viral vector to be used according to the cells to be transduced. In one embodiment, the viral vector is a lentiviral vector or a retroviral vector, such as a gammaretroviral vector. In a further embodiment, the viral vector is a gammaretroviral vector, such as murine stem cell virus (MSCV) or Moloney Murine Leukaemia Virus (MLV). In a yet further embodiment, the viral vector is pseudotyped with an envelope other than vesicular stomatitis virus-G (VSV-G), for example a betaretroviral envelope such as baboon endogenous virus (BaEV) or RD114.

In some embodiments, step (ii) is performed using between 1×10⁶ and 1×10⁸ TU/ml, such as about 1×10⁶, about 5×10⁶, about 1×10⁷, about 5×10⁷ or about 1×10⁸ TU/ml of viral vector. In a particular embodiment, step (ii) is performed using 1×10⁷ TU/ml of viral vector. In other embodiments, step (ii) is performed using an MOI of viral vector between 0.5 and 50, such as an MOI of about 0.5, about 1, about 1.5, about 2.5, about 5, about 10, about 25, about 40 or about 50. In one embodiment, step (ii) is performed using an MOI of viral vector of 2.5. In another embodiment, step (ii) is performed using an MOI of viral vector of 5. In a further embodiment, step (ii) is performed using an MOI of viral vector of 10.

In one embodiment, the tumour associated antigen is an antigen associated with a solid tumour. Thus, in some embodiments the tumour and/or cancer is a solid tumour. Constitutive expression of CD70, a member of the tumour necrosis family, has been described in both haematological and solid cancers where it increases the survival of tumour cells and regulatory T cells within the tumour microenvironment by signalling through its receptor, CD27. Thus, in a further embodiment the solid tumour is a CD70⁺ tumour. It will be appreciated that CD70 may be used to target engineered γδ T cells to said tumours. Therefore, in a yet further embodiment the tumour associated antigen is CD70.

In an alternative embodiment, the tumour associated antigen is mesothelin (also referred to “MSLN” herein). Mesothelin is a 40 kDa protein that is expressed in mesothelial cells and is overexpressed in several tumours, including mesothelioma, ovarian cancer, pancreatic adenocarcinoma, lung adenocarcinoma and cholangiocarcinoma. It has therefore been proposed as a tumour marker or tumour associated antigen which may be targeted in immunotherapy (Hassan et al. Clin. Cancer Res., 2004, 10 (12): 3937-3942). The expression of mesothelin in these tumours may contribute to the implantation and peritoneal spread of tumours by cell adhesion (Rump et al., Biological Chemistry, 2004, 279 (10): 9190-9198).

### Expanded Cell Populations

According to one aspect of the invention, there is provided an expanded γδ T cell population obtained by the methods described herein. According to a further aspect, there is provided an engineered γδ T cell population obtained by the methods described herein.

In some embodiments, the expanded/engineered γδ T cell population comprises greater than 50% γδ T cells, such as greater that 75% γδ T cells, in particular greater that 85% γδ T cells. In one embodiment, the expanded/engineered population comprises Vδ1 cells, wherein less than 50% of the Vδ1 cells express TIGIT. In one embodiment, the expanded/engineered population comprises Vδ1 cells, wherein more than 50%, such as more than 60% of the Vδ1 cells express CD27.

In some aspects, the expanded/engineered γδ T cell population comprises less than about 1% αβ T cells. In some aspects, the expanded/engineered γδ T cell population comprises less than about 1% αβ T cells, less than about 0.9% αβ T cells, less than about 0.8% αβ T cells, less than about 0.7% αβ T cells, less than about 0.6% αβ T cells, less than about 0.5% αβ T cells, less than about 0.4% αβ T cells, less than about 0.3% αβ T cells, less than about 0.2% αβ T cells, less than about 0.1% αβ T cells, less than about 0.09% αβ T cells, less than about 0.08% αβ T cells, less than about 0.07% αβ T cells, less than about 0.06% αβ T cells, less than about 0.05% αβ T cells, less than about 0.04% αβ T cells, less than about 0.05% αβ T cells, less than about 0.04% αβ T cells, less than about 0.03% αβ T cells, less than about 0.02% αβ T cells, less than about 0.01% αβ T cells, less than about 0.009% αβ T cells, less than about 0.008% αβ T cells, less than about 0.007% αβ T cells, less than about 0.006% αβ T cells, less than about 0.005% αβ T cells. In some aspects, the expanded/engineered γδ T cell population comprises less than about 0.9% αβ T cells. In some aspects, the expanded/engineered γδ T cell population comprises less than about 0.8% αβ T cells. In some aspects, the expanded/engineered γδ T cell population comprises less than about 0.7% αβ T cells. In some aspects, the expanded/engineered γδ T cell population comprises less than about 0.6% αβ T cells. In some aspects, the expanded/engineered γδ T cell population comprises less than about 0.5% αβ T cells. In some aspects, the expanded/engineered γδ T cell population comprises less than about 0.4% αβ T cells. In some aspects, the expanded/engineered yd T cell population comprises less than about 0.3% αβ T cells. In some aspects, the expanded/engineered γδ T cell population comprises less than about 0.2% αβ T cells. In some aspects, the expanded/engineered γδ T cell population comprises less than about 0.1% αβ T cells. In some aspects, the expanded/engineered γδ T cell population comprises less than about 0.09% αβ T cells. In some aspects, the expanded/engineered γδ T cell population comprises less than about 0.08% αβ T cells. In some aspects, the expanded/engineered γδ T cell population comprises less than about 0.07% αβ T cells. In some aspects, the expanded/engineered γδ T cell population comprises less than about 0.06% αβ T cells. In some aspects, the expanded/engineered γδ T cell population comprises less than about 0.05% αβ T cells. In some aspects, the expanded/engineered γδ T cell population comprises less than about 0.04% αβ T cells. In some aspects, the expanded/engineered γδ T cell population comprises less than about 0.03% αβ T cells. In some aspects, the expanded/engineered γδ T cell population comprises less than about 0.02% αβ T cells. In some aspects, the expanded/engineered γδ T cell population comprises less than about 0.01% αβ T cells. In some aspects, the expanded/engineered γδ T cell population comprises a concentration of αβ T cells that is below detectable limits.

The expanded/engineered γδ T cell population obtained by the methods described herein may be used as a medicament, for example for adoptive T cell therapy. This involves the transfer of an expanded/engineered population obtained by the methods into a patient. The therapy may be autologous, i.e. the γδ T cells may be transferred back into the same patient from which they were obtained, or the therapy may be allogeneic, i.e. the γδ T cells from one person may be transferred into a different patient. In instances involving allogeneic transfer, the expanded/engineered population may be substantially free of αβ T cells. For example, αβ T cells may be depleted from the expanded/engineered population, e.g. after engineering, using any suitable means known in the art (e.g. by negative selection, e.g. using magnetic beads). A method of treatment may include: providing a sample obtained from a donor individual; expanding and/or engineering the γδ T cells as described herein to produce an expanded/engineered population; and administering the expanded/engineered population of γδ T cells to a recipient individual.

In some aspects, the expanded/engineered γδ T cell population obtained by methods described herein has increased persistence of in vivo cytotoxicity as compared to similarly engineered cells obtained by standard methods. In some aspects, the expanded/engineered γδ T cell population obtained by methods described herein are capable of in vivo cytotoxicity for at least about 7 days, at least about 8 days, at least about 9 days, at least about 10 days, at least about 11 days, at least about 12 days, at least about 13 days, at least about 14 days, at least about 15 days, at least about 16 days, at least about 17 days, at least about 18 days, at least about 19 days, at least about 20 days, or at least about 21 days. In some aspects, the expanded/engineered γδ T cell population obtained by methods described herein are capable of in vivo cytotoxicity for at least about 7 days. In some aspects, the expanded/engineered γδ T cell population obtained by methods described herein are capable of in vivo cytotoxicity for at least about 8 days. In some aspects, the expanded/engineered γδ T cell population obtained by methods described herein are capable of in vivo cytotoxicity for at least about 9 days. In some aspects, the expanded/engineered γδ T cell population obtained by methods described herein are capable of in vivo cytotoxicity for at least about 10 days. In some aspects, the expanded/engineered γδ T cell population obtained by methods described herein are capable of in vivo cytotoxicity for at least about 11 days. In some aspects, the expanded/engineered γδ T cell population obtained by methods described herein are capable of in vivo cytotoxicity for at least about 12 days. In some aspects, the expanded/engineered γδ T cell population obtained by methods described herein are capable of in vivo cytotoxicity for at least about 13 days. In some aspects, the expanded/engineered γδ T cell population obtained by methods described herein are capable of in vivo cytotoxicity for at least about 14 days. In some aspects, the expanded/engineered γδ T cell population obtained by methods described herein are capable of in vivo cytotoxicity for at least about 15 days. In some aspects, the expanded/engineered γδ T cell population obtained by methods described herein are capable of in vivo cytotoxicity for at least about 16 days. In some aspects, the expanded/engineered γδ T cell population obtained by methods described herein are capable of in vivo cytotoxicity for at least about 17 days. In some aspects, the expanded/engineered γδ T cell population obtained by methods described herein are capable of in vivo cytotoxicity for at least about 18 days. In some aspects, the expanded/engineered γδ T cell population obtained by methods described herein are capable of in vivo cytotoxicity for at least about 19 days. In some aspects, the expanded/engineered γδ T cell population obtained by methods described herein are capable of in vivo cytotoxicity for at least about 20 days. In some aspects, the expanded/engineered γδ T cell population obtained by methods described herein are capable of in vivo cytotoxicity for at least about 21 days.

In one embodiment, the expanded/engineered γδ T cell population obtained by methods described herein is for use in the treatment of cancer. “Cancer,” as used herein, refers to the abnormal growth or division of cells. Generally, the growth and/or life span of a cancer cell exceeds, and is not coordinated with, that of the normal cells and tissues around it. Cancers may be benign, pre-malignant or malignant. Cancer occurs in a variety of cells and tissues, including the oral cavity (e.g. mouth, tongue, pharynx, etc.), digestive system (e.g. oesophagus, stomach, small intestine, colon, rectum, liver, bile duct, gall bladder, pancreas, etc.), respiratory system (e.g. larynx, lung, bronchus, etc.), bones, joints, skin (e.g. basal cell, squamous cell, meningioma, etc.), breast, genital system, (e.g. uterus, ovary, prostate, testis, etc.), urinary system (e.g. bladder, kidney, ureter, etc.), eye, nervous system (e.g. brain, etc.), endocrine system (e.g. thyroid, etc.), and haematopoietic system (e.g. lymphoma, myeloma, leukaemia, acute lymphocytic leukaemia, chronic lymphocytic leukaemia, acute myeloid leukaemia, chronic myeloid leukaemia, etc.).

In a further embodiment, the cancer is a solid malignancy (also referred to herein as a solid tumour). Solid tumours can occur in several places, for example in the tissues, bones, muscles and/or organs. It will be understood that a solid tumour does not include cancers of the blood (i.e. haematological cancers). In an alternative embodiment, the cancer is a haematological cancer.

In some aspects, the cancer comprises bone cancer, pancreatic cancer, skin cancer, cancer of the head or neck, cutaneous or intraocular malignant melanoma, lung cancer (e.g., non-small cell lung cancer (NSCLC) or small cell lung cancer (SCLC)), uterine cancer, ovarian cancer, rectal cancer, cancer of the anal region, stomach cancer, testicular cancer, uterine cancer, carcinoma of the fallopian tubes, carcinoma of the endometrium, carcinoma of the cervix, carcinoma of the vagina, carcinoma of the vulva, Hodgkin's Disease, non-Hodgkin's lymphoma, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, chronic or acute leukemia, acute myeloid leukemia (AML), chronic myeloid leukemia, acute lymphoblastic leukemia, chronic lymphocytic leukemia (ALL), chronic myelogenous leukemia, solid tumors of childhood, lymphocytic lymphoma, cancer of the bladder, cancer of the kidney or ureter, carcinoma of the renal pelvis, neoplasm of the central nervous system (CNS), primary CNS lymphoma, tumor angiogenesis, spinal axis tumor, brain stem glioma, pituitary adenoma, Kaposi's sarcoma, epidermoid cancer, squamous cell cancer, T-cell lymphoma, environmentally induced cancers including those induced by asbestos, or any combination thereof. In some aspects, the cancer is locally advanced. In some aspects, the cancer is metastatic. In some aspects, the cancer is refractory. In some aspects, the cancer is relapsed. In some aspects, the cancer is refractory or relapsed following one or more prior anti-cancer therapy. In some aspects, the one or more prior anti-cancer therapies comprise a standard of care therapy.

The patient or subject to be treated is preferably a human cancer patient (e.g. a human cancer patient being treated for a solid tumour) or a virus-infected patient (e.g. a CMV-infected or HIV infected patient). In some instances, the patient has and/or is being treated for a solid tumour. Because they are normally resident in non-haematopoietic tissues, tissue-resident Vδ1 T cells are also more likely to home to and be retained within tumour masses than their systemic blood-resident counterparts and adoptive transfer of these cells is likely to be more effective at targeting solid tumours and potentially other non-haematopoietic tissue-associated immunopathologies.

As γδ T cells are non-MHC restricted, they do not recognise a host into which they are transferred as foreign, which means that they are less likely to cause graft-versus-host disease. This means that they can be used “off the shelf” and transferred into any recipient, e.g. for allogeneic adoptive T cell therapy.

γδ T cells obtained by methods described herein express NKG2D and respond to a NKG2D ligand (e.g. MICA), which is strongly associated with malignancy. They also express a cytotoxic profile in the absence of any activation and are therefore likely to be effective at killing tumour cells. For example, the expanded/engineered γδ T cells obtained as described herein may express one or more, preferably all of IFN-γ, TNF-α, GM-CSF, CCL4, IL-13, Granulysin, Granzyme A and B, and Perforin in the absence of any activation. IL-17A may not be expressed.

The expanded/engineered γδ T cells obtained by the methods described herein may be suitable as an “off-the-shelf” immunotherapeutic reagent. These cells possess innate-like killing, have no MHC restriction and display improved homing to and/or retention within tumours than do other T cells.

In some embodiments, a method of treatment of an individual with a solid tumour in a non-haematopoietic tissue may include: expanding/engineering γδ T cells from a sample from the individual as described herein to produce an expanded/engineered population; and administering the expanded/engineered population of γδ T cells to the individual. In alternative embodiments, the method of treatment comprises expanding/engineering γδ T cells from a sample from a different individual as described herein to produce an expanded/engineered population; and administering the expanded/engineered population of γδ T cells to the individual with a solid tumour. In one embodiment, the amount of expanded/engineered γδ T cells administered to the individual is a therapeutically effective amount.

In further embodiments, the method of treatment and/or the therapeutically effective amount comprises those disclosed in WO2020095058 or WO2020095059, the contents of which are incorporated in their entirety.

Pharmaceutical compositions may include expanded and/or engineered γδ T cells as described herein in combination with one or more pharmaceutically or physiologically acceptable carrier, diluents, or excipients. Such compositions may include buffers such as neutral buffered saline, phosphate buffered saline and the like; carbohydrates such as glucose, mannose, sucrose or dextrans, mannitol; proteins; polypeptides or amino acids such as glycine; antioxidants; chelating agents such as EDTA or glutathione; adjuvants (e.g. aluminium hydroxide); and preservatives. Cryopreservation solutions which may be used in the pharmaceutical compositions of the invention include, for example, DMSO. Compositions can be formulated, e.g. for intravenous administration.

Thus, according to another aspect of the invention, there is provided a pharmaceutical composition comprising the expanded γδ T cell population or the engineered γδ T cell population as described herein.

In one embodiment, the pharmaceutical composition is substantially free of (e.g. there are no) detectable levels of a contaminant, e.g. endotoxin or mycoplasma.

According to a yet further aspect of the invention, there is provided the expanded γδ T cell population, the engineered γδ T cell population or the pharmaceutical composition as described herein for use as a medicament. In another aspect, there is provided the expanded γδ T cell population, the engineered γδ T cell population or the pharmaceutical composition as described herein for use in the treatment of cancer. In a further embodiment, the cancer is a solid tumour.

It will be understood that all embodiments described herein may be applied to all aspects of the invention.

As used herein, the term “about” includes up to and including 10% greater and up to and including 10% lower than the value specified, suitably up to and including 5% greater and up to and including 5% lower than the value specified, especially the value specified. The term “between” includes the values of the specified boundaries.

The practice of the present disclosure will employ, unless otherwise indicated, conventional techniques of cell biology, cell culture, molecular biology, transgenic biology, microbiology, recombinant DNA, and immunology, which are within the skill of the art. Such techniques are explained fully in the literature. See, for example, Sambrook et al., ed. (1989) Molecular Cloning A Laboratory Manual (2nd ed.; Cold Spring Harbor Laboratory Press); Sambrook et al., ed. (1992) Molecular Cloning: A Laboratory Manual, (Cold Springs Harbor Laboratory, NY); D. N. Glover ed., (1985) DNA Cloning, Volumes I and II; Gait, ed. (1984) Oligonucleotide Synthesis; Mullis et al. U.S. Pat. No. 4,683,195; Hames and Higgins, eds. (1984) Nucleic Acid Hybridization; Hames and Higgins, eds. (1984) Transcription And Translation; Freshney (1987) Culture Of Animal Cells (Alan R. Liss, Inc.); Immobilized Cells And Enzymes (IRL Press) (1986); Perbal (1984) A Practical Guide To Molecular Cloning; the treatise, Methods In Enzymology (Academic Press, Inc., N.Y.); Miller and Calos eds. (1987) Gene Transfer Vectors For Mammalian Cells, (Cold Spring Harbor Laboratory); Wu et al., eds., Methods In Enzymology, Vols. 154 and 155; Mayer and Walker, eds. (1987) Immunochemical Methods In Cell And Molecular Biology (Academic Press, London); Weir and Blackwell, eds., (1986) Handbook Of Experimental Immunology, Volumes I-IV; Manipulating the Mouse Embryo, Cold Spring Harbor Laboratory Press, Cold Spring Harbor, N.Y., (1986)); Crooke, Antisense drug Technology: Principles, Strategies and Applications, 2nd Ed. CRC Press (2007) and in Ausubel et al. (1989) Current Protocols in Molecular Biology (John Wiley and Sons, Baltimore, Md.).

All of the references cited above, as well as all references cited herein, are incorporated herein by reference in their entireties.

Certain aspects and embodiments of the invention will now be illustrated by way of the following examples and with reference to the figures described above.

### EXAMPLES

### Example 1. Materials and Methods

### Cell Culture

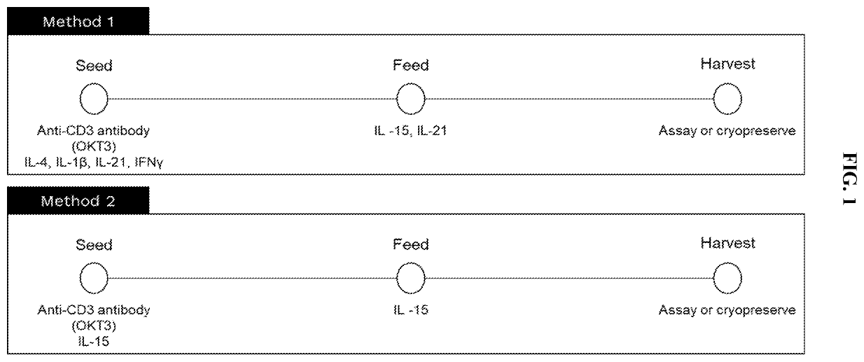

Vδ1+γδ T cell enriched cultures were generated according to the methods described below and as summarized in FIG. 1.

Method 1: Vδ1+γδ T cells were generated from αβ-TCR+ depleted peripheral blood mononuclear cells using serum-free culture medium (CTS OpTmizer, Thermo Fisher) supplemented with 2.5% pooled allogeneic plasma (Octaplas, Octapharma) and Glutamax (ThermoFisher). The isolated cells were seeded in the presence of recombinant IL-4 (Miltenyi), IL-1β (Miltenyi), IL-21 (CellGenix), IFNγ (Bio-Techne) and soluble anti-CD3 (clone OKT3) antibody (Biolegend). After setup, cultures were incubated at 37° C. and 5% CO₂ in a humidified incubator. Culture media was supplemented with fresh IL-21, 39 ng/ml, (CellGenix) and IL-15, 21 ng/ml (Bio-Techne) on day 7 and IL-15, 100 ng/ml (Bio-Techne) day 11. Cells were harvested after 14 days of culture and cryopreserved as a mixed population in Cryostor5 (STEMCELL Technologies).

Method 2: Vδ1+γδ T cells were generated from αβ-TCR+ depleted peripheral blood mononuclear cells using serum-free culture medium (CTS OpTmizer, Thermo Fisher) supplemented with 2.5% pooled allogeneic plasma (Octaplas, Octapharma) and Glutamax (ThermoFisher). The isolated cells were seeded in the presence of recombinant IL-15, 100 ng/ml (Bio-Techne) and soluble anti-CD3 (clone OKT3) antibody (Biolegend), without the presence of, e.g. in absence of, IL-21. After setup, cultures were incubated at 37° C. and 5% CO₂ in a humidified incubator. Expanding cells were fed with fresh IL-15, 100 ng/ml (Bio-Techne). Cells were harvested after 14 days of culture and cryopreserved as a mixed population in Cryostor5 (STEMCELL Technologies).

Flow cytometry assessment of cell purity and surface phenotype

Standard Characterization: Immunophenotyping was performed using a MACSQuant16 or BD FACSLyric™ flow cytometry instruments. The same protocol was applied to characterize both cell products at harvest and post-cryopreservation. Dead cells were excluded using LIVE/DEAD™ Fixable Aqua Dead Cell Stain kit (Invitrogen). Cells were analyzed for the expression of surface markers using BV421-CD27, BV421-DNAM-1, BV605-PD-1, FITC-Mesothelin, PE-NKG2D, PE-NKp30, PE-Cy7-CD56, PE-Cy7-TIGIT, APC-TCRγδ, APC-Cy7-Vδ1 antibodies, available from Miltenyi, BioLegend and ACRO Biosystems.

Deep Characterization: Deep immunophenotype characterization was performed using Biolegend's LEGENDScreen™ Human PE kit (#700007). This deep characterization involved the investigation of the cell surface expression of 361 markers in cell products generated according to Method 1 or 2.

### Quantification of Secreted Factors:

Secreted proteins were quantified post-cryopreservation. Briefly, Vδ1+γδ T cell products were thawed and co-cultured overnight with OVCAR3 adherent tumour targets. Then, supernatants were collected and labelled as per manufacturer's instructions using the 65-Plex Human ProcartaPlex™ Panel (Cat no: EPX650-10065-901). Labelled samples were analysed using the Luminex® FLEXMAP 3D platform. This assay allowed for the simultaneous detection and quantification of 65 secreted proteins, including cytokines and chemokines, during the co-culture with target cells. Effectors alone were used as controls.

Transduction with gammaretroviral vector encoding anti-MSLN chimeric antigen receptor

Cells were expanded according to Method 1 or 2 and transduced with a γ-retroviral vector encoding for Mesothelin (MSLN) targeting chimeric antigen receptor in the presence of RetroNectin (20 μg/mL). Viral vector was mixed with immune cells diluted in CTS OpTmizer overnight at 37° C. Transduction efficiency was determined by flow cytometry four to nine days post-transduction.

### Thawing Cryopreserved Cell Products

Frozen cryovials were thawed in a 37° C. water bath and added to pre-warmed OpTmizer+2.5% allogeneic plasma. Cells were spun down at 300 g for 7 minutes, counted and viability assessed, and then resuspended at 2×10⁶ cells per mL for phenotyping and downstream assays.

### Cytotoxicity Assays

Expanded cells were co-cultured with adherent tumour targets (e.g. ovarian adenocarcinoma cell line, OvCAR3) some of which expressing firefly luciferase, at various effector to target ratios. Target cells in the absence of effectors served as a control (Ctrl). Maximum lysis was determined using Staurosporine treated target cells. After 20 hours, quantification of target viability was assessed using ONE-Glo™ Luciferase Assay System kit (Promega) or CellTiter-Glo® Luminescent Cell Viability Assay (Promega). Luminescence was measured on Biotek Synergy H4 plate reader.

### Repeated Antigen Stimulation Assay

Cryopreserved Vδ1+γδ T cell products were thawed and co-cultured in presence of cytokines with adherent tumour targets expressing GFP (e.g. lung cancer cell line, A549-Meso+) at an effector to target ratio of 2.5 effectors for each target. Cells were incubated at 37° C. and 5% CO₂ in an Incucyte® Live-Cell Analysis System (Sartorius). Vδ1+γδ T cell products were challenged by adding fresh tumor targets to the culture every 3-4 days. % of GFP was used as a measure of residual tumor targets at each time point across the assay. To calculate Vδ1+γδ fold expansion, cells were counted at each time point and flow cytometry was performed to determine Vδ1+γδ enrichment.

### Example 2. Cell Product Comparison

αβ-TCR+ depleted peripheral blood mononuclear cells were expanded using either Method 1 or 2 described in Example 1 and as summarized in FIG. 1. The expanded cell products were then analyzed and compared.

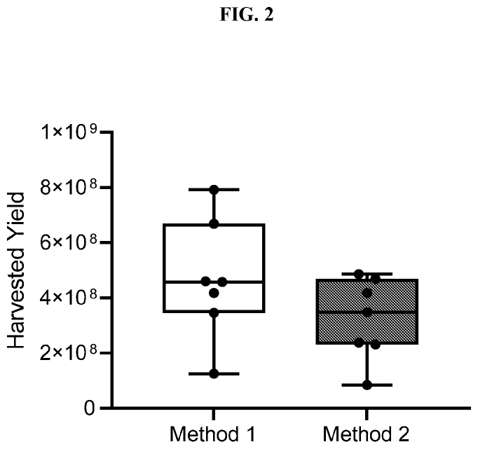

Cell yields at harvest are shown in FIG. 2. Growing the cells according to Method 2 resulted in comparable (or slightly lower) cell yields to Method 1. Method 2 resulted in reduced donor-to-donor yield variability.

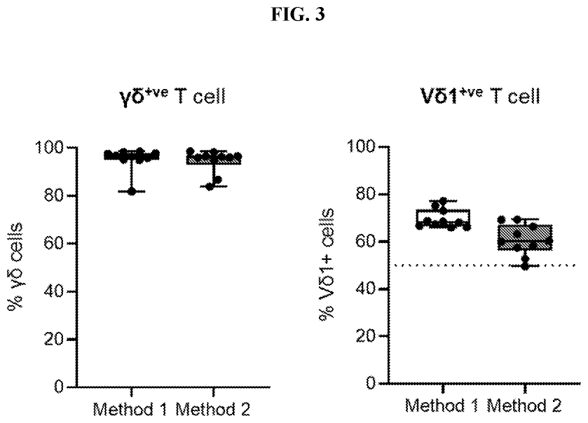

Immune cell composition was analyzed by flow cytometry (FIG. 3). Both expansion methodologies resulted in pure γδ T cell products, highly enriched for Vδ1 T cells (>50%).

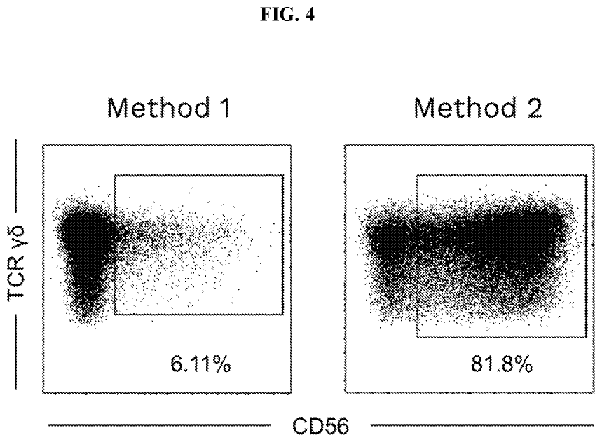

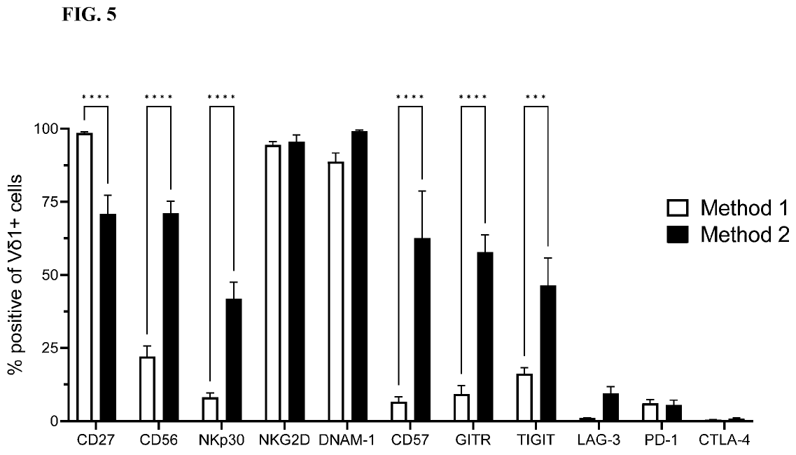

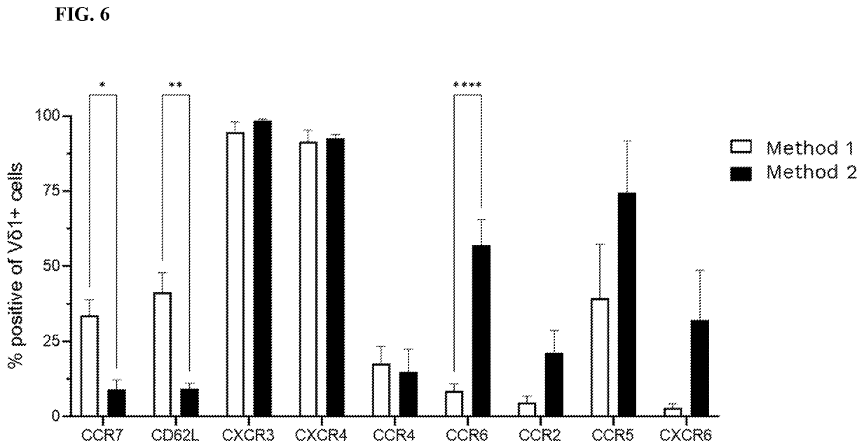

Vδ1 T cells produced using Method 2 upregulated CD56, a key marker of the cell product generated by this method. Representative flow plots are shown in FIG. 4. Vδ1 T cells expanded by Method 2 presented a distinct cell surface marker expression with significantly lower expression of CD27 and higher expression of CD56, NKp30, CD57, GITR and TIGIT, consistent with a more effector-like phenotype. Markers associated with exhaustion such as LAG-3, PD-1 and CTLA-4 remained low or undetectable (FIG. 5). Furthermore, expanding the cells according to Method 2 altered the expression of various chemokine receptors (FIG. 6). Whilst CD62L and CCR7 (two markers associated with lymph node retention) were significantly downregulated, CCR6 (a marker associated with peripheral retention) was significantly upregulated.

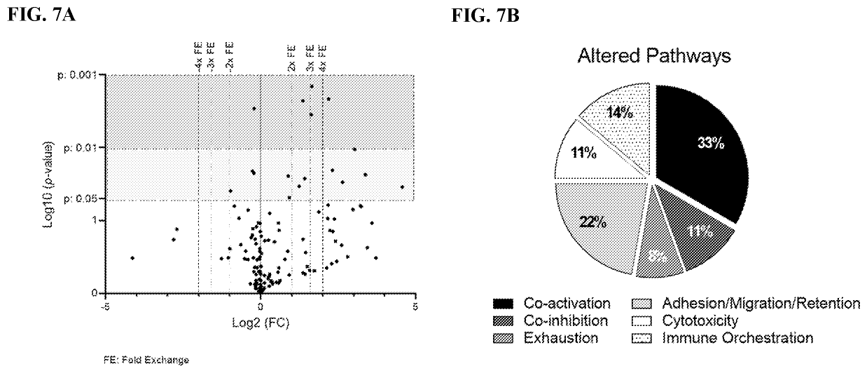

Cell products generated by Method 1 and 2 were deep phenotyped using LegendScreen technology, which allows for the simultaneous analysis of 364 cell surface markers. Flow cytometry results were analysed using ANOVA and represented as Volcano plots. Analysis revealed distinct differentially expressed surface markers between the two expansion methods (FIG. 7A). Each individual dot in the volcano plot represents an individual cell surface marker analyzed. The higher the position of that dot within the y axis the greater the significance of the differential expression of that marker between the cells generated by Method 2 when compared to Method 1. The more decentralized position of that dot within the x axis (higher or lower than 0), the greater the fold exchange expression of that individual marker between cells generated by Method 2 when compared to Method 1. The cell surface markers significantly altered by growing the cells according to Method 2 when compared to Method 1 are mainly involved in co-activation, adhesion/migration/retention and immune orchestration signaling pathways (FIG. 7B). The most relevant and significantly altered markers are listed in a table format in Table 1 below. In summary, the altered marker expression contributes to the acquisition of a distinct functionality of the cells generated by Method 2 when compared to Method 1.

TABLE 1
Relevant and Significantly altered markers

| Marker | Up/Down-Regulated | Significant? |
|---|---|---|
| SLAMF1 | Up | Yes |
| NKG2 | Up | Yes |
| FcεR1α | Up | Yes |
| Integrin alpha Z | Down | Yes |
| CD86 | Up | Yes |
| CD70 | Up | Yes |
| IL-2Ra | Up | Yes |
| CD9 | Down | Yes |
| CD6 | Down | Yes |
| CEACAM 1/6/5 | Up | Yes |
| Integrin alpha 2 | Up | Yes |
| CD85 | Up | Yes |
| GITR | Up | Yes |
| TIGIT | Up | Yes |
| CD16 | Up | Yes |
| OX2R | Down | Yes |
| CCR6 | Up | Yes |
| CCR5 | Up | Yes |
| Tim-3 | Up | Yes |
| LAG3 | Up | Yes |
| CD57 | Up | Yes |
| CD80 | Up | Nearly |
| CD27 | Down | Yes |
| CD56 | Up | Yes |
| CCR7 | Down | Yes |
| CD62L | Down | Yes |
| CCR2 | Up | Nearly |
| NKp30 | Up | Yes |
| CD59 | Up | Nearly |
| Integrin alpha L | Down | Nearly |

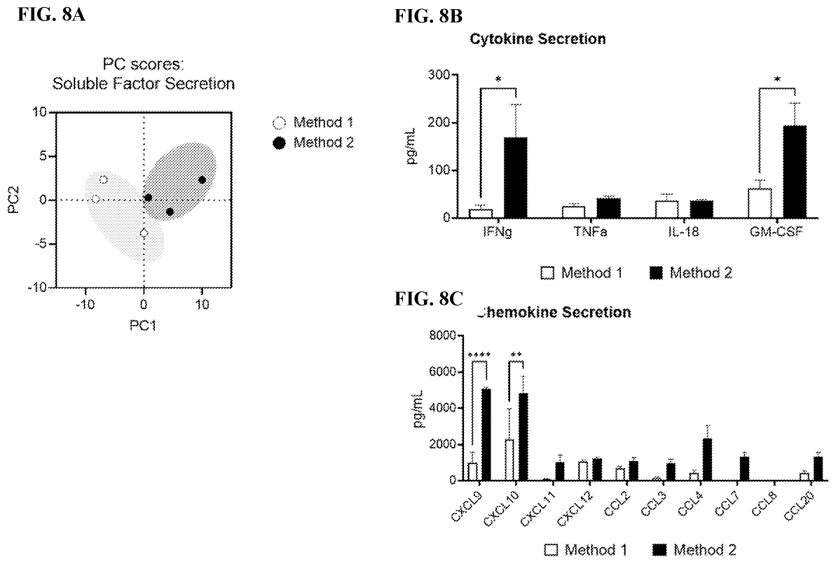

Cell products generated by Method 1 and 2 were analysed for factor secretion using Luminex technology, which allows for the simultaneous detection and quantification of 65 proteins per sample. Principal Component Analysis (PCA) was performed (FIG. 8A) to measure variance in soluble factor secretion between samples prepared with Method 1 and Method 2. PCA analysis indicated that samples generated with Method 2 (black dots) cluster separately from samples generated from Method 1 (white dots), revealing a distinct soluble factor secretion profile for γδ T cell products generated by Method 2 when compared to Method 1. Vδ1 T cells expanded by Method 2 presented a significant increase of secretion of effector cytokines such as IFNγ and GM-CSF compared to Method 1 (FIG. 8B). Additionally, Method 2 resulted in a significant increase of secretion of chemokine such as CXCL9 and CXCL10 and nearly significant increase of CXCL11, CCL4, CCL7, CCL20 (FIG. 8C).

### Example 3. Cell Product Functionality after Storage

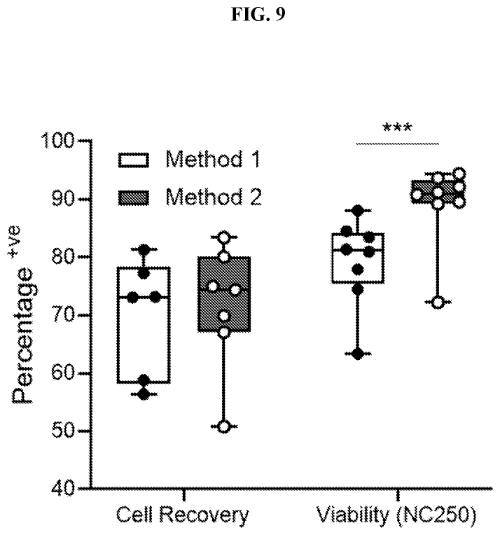

The functionality of the cells was also investigated. Frozen cell products were thawed according to Example 1. Total cell recovery and viability post-cryopreservation were comparable between the cell products generated using Method 1 or 2. Nonetheless, cells generated according to Method 2 presented slightly higher viability post-cryopreservation (FIG. 9). Importantly, immune cell composition and cell surface phenotype of the cell products were retained post-cryopreservation (data not shown).

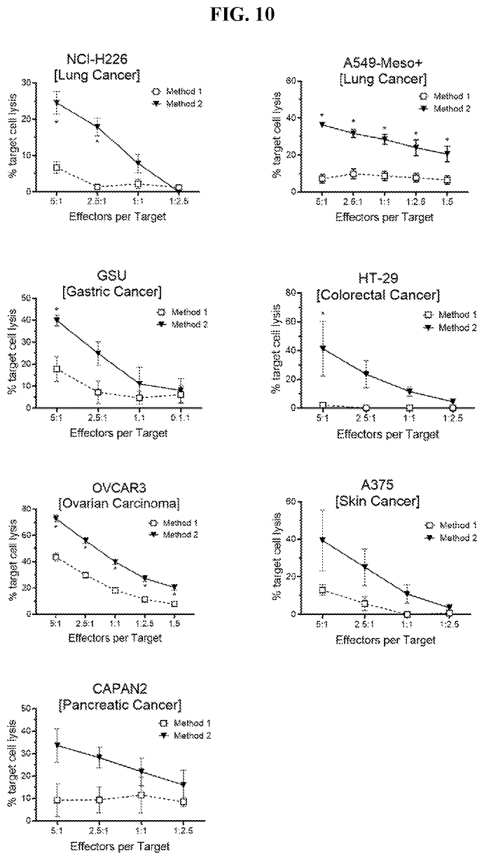

Cytotoxicity of cell products post-cryopreservation was also investigated. Cell products generated according to Method 2 were significantly more cytotoxic against a wide array of solid tumor cancer cell lines across a range of effector to target ratios, relative to cells generated using Method 1 (FIG. 10).

### Example 4. Cell Product Functionality after Transduction

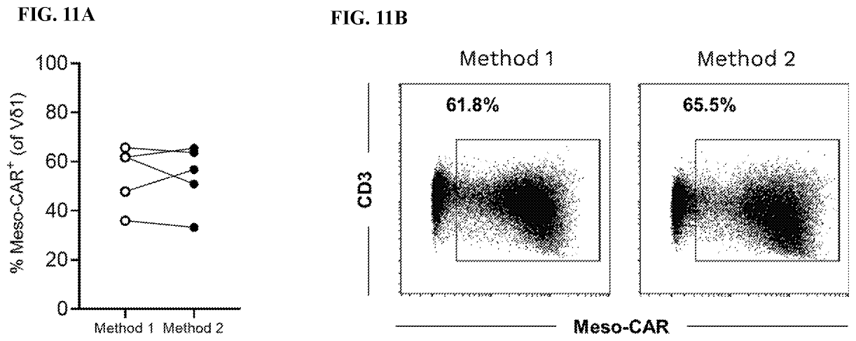

Expanding cells were transduced with γ-retroviral vector as described in Example 1 and gene transfer efficiencies were determined by flow cytometry. Chimeric antigen receptor expression was identical between cells expanded by Method 1 or 2, suggesting modifying the expansion method does not alter the permissiveness of Vδ1 T cells for gammaretroviral transduction (FIG. 11A). A representative flow characterization of CAR expression in cells transduced and expanded by Method 1 or 2 can be seen in FIG. 11B.

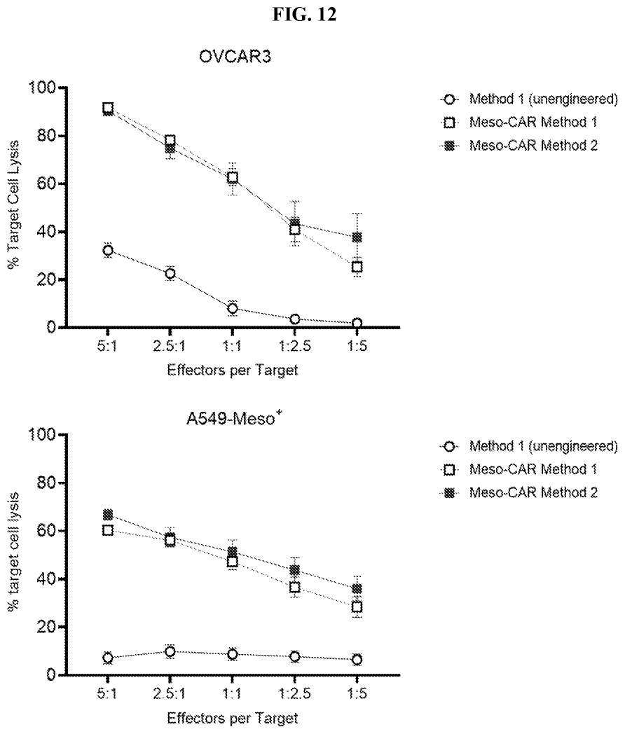

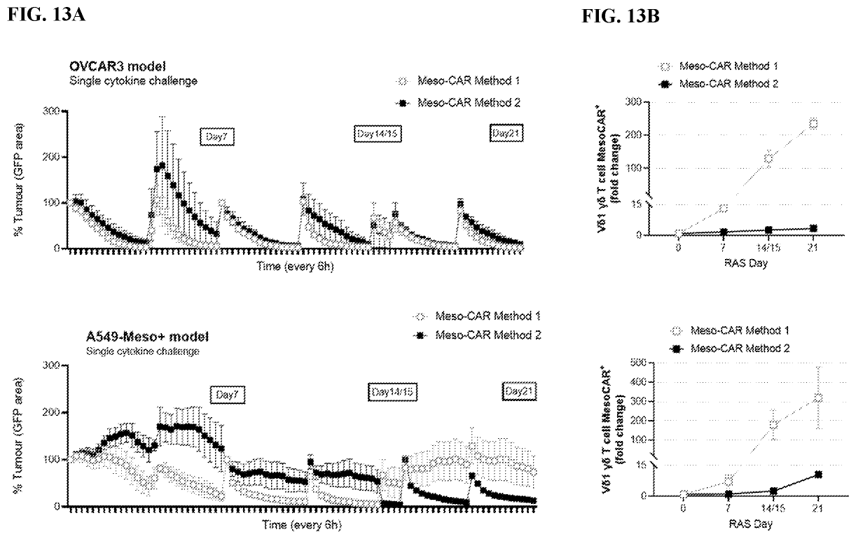

Furthermore, the functionality of the engineered cell products was also investigated in an overnight cytotoxicity assay (FIG. 12) and repeated tumor challenging (FIG. 13) in two different models using OVCAR3 and A549-Meso tumor cell lines. In the context of an overnight cytotoxicity assay, CAR transduced cells exhibited comparable cytotoxicity regardless the method to expand them. CAR transduction greatly potentiated overnight cytotoxicity of cell products expanded according to Method 1 (FIG. 12). However, in the context of a 21-day Repeated Antigen Stimulation (RAS) assay, where our cell products are challenged with tumor every 3 to 4 days, only CAR transduced cells expanded with Method 2 were capable to control tumor growth until Day 21 in all models used (FIG. 13A). Additionally, the superior tumor control exhibited by the products expanded by Method 2 was possible with a significantly lower fold expansion during the assay when compared to cell products grown with Method 1 (FIG. 13B). This is indicative that cell products grown with Method 2 are much more capable of multiple effective rounds of serial killing and control tumor over time. All together, these observations emphasize that empowering Vδ1 T cells with innate cytotoxic functions against tumor targets synergizes with engineering strategies. Therefore, the Method 2 culture process enhances functionality of γδ cell products ahead and independently of engineering.

### Example 5. Analysis of IL-15 Concentration

Vδ1+γδ T cells will be generated from αβ-TCR+ depleted peripheral blood mononuclear cells as described in example 1. The isolated cells will be seeded in the presence of recombinant IL-15 at various concentrations and soluble anti-CD3 (clone OKT3) antibody (Biolegend), without the presence of, e.g. in absence of, IL-21. After setup, cultures will be incubated at 37° C. and 5% CO₂ in a humidified incubator. Expanding cells will be fed with fresh IL-15at various concentrations. Cells will be harvested after 14 days of culture and cryopreserved for further analysis. Concentrations of IL-15 that will be tested include, but are not limited to, from about 5 ng/mL to about 150 ng/ml (e.g., 5 ng/ml, 10 ng/mL, 15 ng/mL, 20 ng/mL, 25 ng/mL, 30 ng/ml, 35 ng/mL, 40 ng/mL, 45 ng/mL, 50 ng/ml, 60 ng/ml, 70 ng/mL, 80 ng/mL, 90 ng/ml, 100 ng/mL, 110 ng/mL, 120 ng/mL, 130 ng/ml, 140 ng/ml, and 150 ng/ml).

1. A method for expanding γδ T cells, wherein said method comprises the steps of:
(1) preparing a composition enriched for γδ T cells by depleting αβ T cells from a sample obtained from a subject which comprises γδ T cells;
(2) culturing the composition enriched for γδ T cells in the presence of:
(i) an anti-CD3 antibody or fragment thereof; and
(ii) Interleukin-15 (IL-15), in the absence of Interleukin-4 (IL-4), from the first day of said culturing; and
(3) isolating the cell population cultured from the composition.

2. The method according to claim 1, wherein the sample is a haematopoietic sample or a fraction thereof.

3. The method according to claim 2, wherein the sample is selected from peripheral blood, umbilical cord blood, lymphoid tissue, thymus, bone marrow, spleen, lymph node tissue or fractions thereof, in particular peripheral blood or a fraction thereof.

4. The method according to claim 3, wherein the sample consists of peripheral blood mononuclear cells (PBMCs) or low density mononuclear cells (LDMCs).

5. The method according to claim 1, wherein the sample is a non-haematopoietic tissue.

6. The method according to any one of claims 1 to 5, wherein the subject is human.

7. The method according to any one of claims 1 to 6, wherein the method comprises culturing the composition for between 7 and 21 days.

8. The method according to claim 7, wherein the method comprises culturing the composition for about 10, 11, 12, 13, or 14 days.

9. The method according to any one of claims 1 to 8, wherein the anti-CD3 antibody is OKT3.

10. The method according to any one of claims 1 to 9, wherein expanding the population of γδ T cells provides at least a 5-fold, especially at least a 10-fold, in particular at least a 20-fold number of γδ T cells.

11. The method according to any one of claims 1 to 10, wherein at least 50% of the expanded γδ T cells present in the cell population express CD56.

12. The method according to any one of claims 1 to 11, wherein the cell population comprises γδ T cells that express NKp30, CD57, GITR, TIGIT, CCR6, CCR2, CCR5 and/or CXCR6.

13. The method according to any one of claims 1 to 12, wherein the γδ T cells are derived from a single donor.

14. The method according to any one of claims 1 to 12, wherein the γδ T cells are derived from multiple donors.

15. The method according to any one of claims 1 to 14, wherein the method comprises freezing the expanded γδ T cells.

16. A method for engineering γδ T cells, said method comprising the steps of:
(i) preparing a composition enriched for γδ T cells using the method according to any one of claims 1 to 15;
(ii) transducing the composition with an exogenous nucleic acid for expression in the γδ T cells; and
(iii) culturing the transduced composition to expand the engineered γδ T cells.

17. The method according to claim 16, wherein the exogenous nucleic acid encodes a chimeric antigen receptor (CAR) recognizing a tumour antigen.

18. The method according to claim 17, wherein the tumour antigen is a tumour specific antigen that is not expressed by normal somatic cells from the subject tissue.

19. The method according to claim 17 or claim 18, wherein the tumour antigen is a tumour associated antigen which is preferentially overexpressed on cancer cells compared to healthy somatic cells.

20. The method according to any one of claims 17 to 19, wherein the tumour antigen is an antigen expressed in the context of stress events such as oxidative stress, DNA damage, UV radiation, EGF receptor stimulation.

21. The method according to any one of claims 17 to 20, wherein the tumour antigen is an antigen associated with a solid tumour.

22. The method according to any one of claims 16 to 21, wherein the composition is transduced using a viral vector, such as a retroviral vector, such as a gammaretroviral vector or a lentiviral vector.

23. The method according to claim 22, wherein the viral vector is a gammaretroviral vector, such as murine stem cell virus (MSCV) or Moloney Murine Leukaemia Virus (MLV).

24. The method according to claim 22 or claim 23, wherein the viral vector is pseudotyped with an envelope other than vesicular stomatitis virus-G (VSV-G), for example a betaretroviral envelope such as baboon endogenous virus (BaEV) or RD114.

25. The method according to any one of claims 16 to 24, wherein step (iii) comprises culturing the transduced composition in the absence of feeder cells.

26. The method according to any one of claims 16 to 24, wherein step (iii) comprises culturing the transduced composition in the presence of feeder cells.

27. An expanded γδ T cell population obtainable, such as obtained, by the method of any one of claims 1 to 26.

28. A pharmaceutical composition comprising the expanded γδ T cell population according to claim 27.

29. The expanded γδ T cell population according to claim 27 or the pharmaceutical composition according to claim 28 for use as a medicament.

30. The expanded γδ T cell population according to claim 27 or the pharmaceutical composition according to claim 28 for use in the treatment of cancer.

31. The expanded γδ T cell population or the pharmaceutical composition for use according to claim 30, wherein the cancer is a solid tumour.

32. The expanded γδ T cell population according to claim 27 or the pharmaceutical composition according to claim 28, wherein the expanded γδ T cells are capable of in vivo cytotoxicity for at least about 7 days, at least about 8 days, at least about 9 days, at least about 10 days, at least about 11 days, at least about 12 days, at least about 13 days, at least about 14 days, at least about 15 days, at least about 16 days, at least about 17 days, at least about 18 days, at least about 19 days, at least about 20 days, or at least about 21 days.

33. The expanded γδ T cell population according to claim 27 or the pharmaceutical composition according to claim 28, wherein the expanded γδ T cells are capable of in vivo cytotoxicity for at least about 14 days.
